(19) United States
(12) Patent Application Publication
(10) Pub. No.: US 2011/0293524 A1
(43) Pub. Date: Dec. 1, 2011

(54) METHODS AND COMPOSITIONS FOR THE REGULATION OF LECTIN COMPLEMENT PATHWAY (LCP)-ASSOCIATED COMPLEMENT ACTIVATION IN HYPERGLYCEMIC MYOCARDIAL DAMAGE

(75) Inventor: Gregory L. Stahl, Clinton, MA (US)

(73) Assignee: The Brigham and Women"s Hosptial Inc., Boston, MA (US)

(21) Appl. No.: 12/920,176

(22) PCT Filed: Feb. 29, 2008
(86) PCT No.: PCT/US08/02689

§ 371 Date: May 5, 2011

(51) Int. Cl. A61K 49/00; C12Q 1/02; A61K 38/02; A61P 9/10; A61K 39/395; A61P 9/00; A61P 43/00; C12N 5/071; A61K 31/713

(52) U.S. Cl. 424/9.2; 435/375; 435/29; 514/16.4; 514/44 A; 424/152.1; 514/1.1

(57) ABSTRACT

The present invention relates to methods and compositions for regulating lectin complement pathway (LCP)-associated complement activation. In particular, the invention relates to methods and compositions for inhibiting LCP-associated complement activation in order to inhibit hyperglycemic myocardial damage. The invention also relates to the treatment of cardiomyopathy and/or hypertrophy, such as cardiac hypertrophy. The invention also relates to methods and compositions for inhibiting the loss of cardiac progenitor cells by inhibiting LCP-associated complement activation. The methods include both in vitro and in vivo methods. The methods can be accomplished by contacting a mammalian cell having a surface exposed mannose binding lectin (MBL) ligand, such as a cardiac cell, with an effective amount of a MBL inhibitor to inhibit LCP-associated complement activation.

### GOVERNMENT SUPPORT

This invention was made with government support under grant numbers HL56068, HL52886, HL79758, DE016191 and DE017821 awarded by the National Institutes of Health. Accordingly, the government has certain rights in the invention.

### FIELD OF THE INVENTION

The present invention relates to methods and compositions for regulating lectin complement pathway (LCP)-associated complement activation. In particular, the invention relates to methods and compositions for inhibiting LCP-associated complement activation in order to inhibit hyperglycemic myocardial damage. The invention also relates to the treatment of cardiomyopathy and/or hypertrophy, such as cardiac hypertrophy. The invention also relates to methods and compositions for inhibiting the loss of cardiac progenitor cells by inhibiting LCP-associated complement activation.

### BACKGROUND OF THE INVENTION

The immune system functions to defend the body against pathogenic bacteria, viruses and parasites. Immunity against foreign pathogens usually involves the complement system. The complement system is a cascade of 18 sequentially activated serum proteins which functions to recruit and activate other cells of the immune system, effect cytolysis of target cells and induce opsonization of foreign pathogens. Complement can be activated by the presence of either antibody/antigen complexes, as in the classical complement pathway, or microbial surfaces, as in the alternative complement pathway. Complement activation can also occur via the lectin complement pathway. Lectins are carbohydrate-binding proteins that recognize oligosaccharide structures present on cell surfaces, the extracellular matrix and secreted glycoproteins. These distinct activation pathways ultimately converge at the common enzymatic step of serum protein C3 cleavage to C3b and C3a. This, in turn, initiates the terminal steps of complement function including the cleavage of C5 to C5b and C5a and subsequent deposition of C5b-C9 onto the target cell membrane.

The LCP is an antibody-independent cascade that is initiated by binding of mannose (or mannan) binding lectin (MBL) to cell surface carbohydrates on bacteria, yeasts, parasitic protozoa and viruses (Turner M W, Immunol. Today, 1996; 17:532-540). MBL (≈600 kDa) is a member of the collectin protein family and is structurally related to the classical complement C1 subcomponent, C1q. Associated with MBL are two serine proteases, mannose binding lectin associated serine protease-1 and mannose binding lectin associated serine protease-2, MASP-1 and MASP-2, respectively, which show striking homology to the two C1q-associated serine proteases of the classical complement pathway, C1r and C1s (Thiel S, et al., Nature 1997; 386:506-510). The selectivity of MBL sugar binding is: N-acetyl-D-glucosamine (GluNAc)>mannose>N-acetylmannosamine and fucose>maltose>glucose>>galactose and N-acetylgalactosamine (Thiel S, et al., Nature 1997; 386:506-510; Turner M W, Immunol. Today, 1996; 17:532-540). Binding of the MBL/MASP complex to cell surface carbohydrates activates the LCP, which in turn activates the classical complement pathway independently of C1q, C1r, C1s or antibodies. Most if not all the carbohydrate moieties to which MBL binds are not normally expressed by unperturbed human tissue.

### SUMMARY OF THE INVENTION

It has been discovered that MBL deficiency is protective in hyperglycemic and diabetic subjects in the absence of ischemia/reperfusion. Therefore, various methods and compositions for inhibiting lectin complement pathway (LCP)-associated complement activation, for example, to inhibit tissue damage that results from the hyperglycemic state are provided.

In one aspect, a method for inhibiting hyperglycemic myocardial damage is provided. In one embodiment, the method includes the step of contacting cardiac cells with an effective amount of a mannose binding lectin (MBL) inhibitor to inhibit lectin complement pathway (LCP)-associated complement activation so as to inhibit hyperglycemic myocardial damage. In another embodiment, the contacting is carried out by administering the MBL inhibitor to a subject in need thereof. In yet another embodiment, the subject is hyperglycemic. In still another embodiment, the subject has diabetes. In one embodiment, the subject has Type I or Type II diabetes. In another embodiment, the contacting is carried out in vitro. In a further embodiment, the cardiac cells are cardiac fibroblasts, endothelial cells, mast cells or vascular smooth muscle cells.

In another aspect, a method for inhibiting lectin complement pathway (LCP)-associated complement activation in a subject with hypertrophy is provided. In one embodiment, the method includes the step of administering an effective amount of a mannose binding lectin (MBL) inhibitor to inhibit LCP-associated complement activation in the subject. In a further embodiment, the amount to inhibit LCP-associated complement activation is effective to treat the hypertrophy. In another embodiment, the hypertrophy is nonischemic hypertrophy. In yet another embodiment, the nonischemic hypertrophy is nonischemic cardiac hypertrophy. In still another embodiment, the nonischemic cardiac hypertrophy is left ventricle hypertrophy. In another embodiment, the subject with nonischemic cardiac hypertrophy is hyperglycemic. In still another embodiment, the subject with nonischemic cardiac hypertrophy has diabetes. In one embodiment, the diabetes is Type I or Type II diabetes.

In still another aspect, a method for inhibiting lectin complement pathway (LCP)-associated complement activation in a subject with cardiomyopathy is provided. In one embodiment, the method includes administering an effective amount of a mannose binding lectin (MBL) inhibitor to inhibit LCP-associated complement activation in the subject. In a further embodiment, the amount to inhibit LCP-associated complement activation is effective to treat the cardiomyopathy. In another embodiment, the cardiomyopathy is nonischemic cardiomyopathy. In still another embodiment, the nonischemic cardiomyopathy is dilated cardiomyopathy, hypertrophic cardiomyopathy, arrhythmogenic right ventricular cardiomyopathy or restrictive cardiomyopathy. In one embodiment, the subject is hyperglycemic. In another embodiment, the subject has diabetes. In one embodiment, the subject has Type I or Type II diabetes.

In yet another aspect, method for inhibiting loss of cardiac progenitor cells is provided. In one embodiment, the method includes the step of contacting cardiac cells with an effective amount of a mannose binding lectin (MBL) inhibitor to inhibit cardiac progenitor cell loss. In another embodiment, the loss of cardiac progenitor cells is hyperglycemic induced. In yet another embodiment, the contacting is carried out by administering the MBL inhibitor to a subject in need thereof. In one embodiment, the subject is hyperglycemic. In another embodiment, the subject has diabetes. In one embodiment, the subject has Type I or Type II diabetes. In another embodiment, the contacting is carried out in vitro. In a further embodiment, the cardiac cells are cardiac fibroblasts, endothelial cells, mast cells or vascular smooth muscle cells.

In a further aspect, a method for inhibiting lectin complement pathway (LCP)-associated complement activation in a diabetic subject is provided. In one embodiment, the method includes the step of administering an effective amount of a mannose binding lectin (MBL) inhibitor to inhibit LCP-associated complement activation in the subject. In a further embodiment, the amount to inhibit LCP-associated complement activation is effective to treat the diabetes. In another embodiment, the diabetic subject is a nonischemic diabetic subject. In yet another embodiment, the subject has Type I or Type II diabetes. In still another embodiment, the subject has diabetic nephropathy. In a further embodiment, the amount to inhibit LCP-associated complement activation is effective to inhibit myocardial damage. In another embodiment, the myocardial damage is cardiac hypertrophy and/or cardiomyopathy.

In one embodiment of any of the methods and compositions provided, the MBL inhibitor binds to MBL, a mannose-binding lectin-associated serine protease (MASP) or mannose. In another embodiment, the MASP is MASP-1 or MASP-2. In yet another embodiment, the MBL inhibitor is an inhibitor that binds MASP-2 or otherwise interferes with LCP-associated complement activation via MASP-2. In a further embodiment, the MBL inhibitor is an inhibitor that binds MASP-1 or otherwise interferes with LCP-associated complement activation via MASP-1. In still a further embodiment, the MBL inhibitor binds or otherwise intereferes with MASP-1 and MASP-2. In another embodiment, the MBL inhibitor binds to a MBL ligand. In still another embodiment, the MBL inhibitor that binds to a MBL ligand is a legume derived lectin or a fragment thereof. In yet another embodiment, the MBL inhibitor that binds to a MBL ligand is a keratin binding molecule. In one embodiment, the keratin binding molecule is an anti-keratin antibody or an antigen-binding fragment thereof.

In yet another embodiment, the MBL inhibitor is a C1 inhibitor. In another embodiment, the MBL inhibitor inhibits C3b deposition. In yet another embodiment, the MBL inhibitor that inhibits C3b deposition with an EC50 of between 10⁻⁹ to 10⁻⁷ mol/L.

In one embodiment, the MBL inhibitor is a peptide, protein, small molecule or siRNA. In another embodiment, the MBL inhibitor is an antibody or antigen-binding fragment thereof. In yet another embodiment, the antibody is a monoclonal antibody. In still another embodiment, the antigen-binding fragment a single-chain antibody, diabody, F(ab′)₂ fragment, Fd fragment, Fv fragment or Fab fragment. In a further embodiment, the antibody is a humanized antibody. In still a further embodiment, the antibody is a human antibody.

The MBL inhibitor may be administered to the subject by any route known in the art. The MBL inhibitor may be administered to the subject by an aerosol route of delivery.

In yet a further aspect, methods of evaluating a candidate MBL inhibitor are provided. In one embodiment, the method includes the steps of selecting a candidate MBL inhibitor, and assessing its ability to treat hypertrophy in a subject. In another embodiment, the step of assessing includes administering the candidate MBL inhibitor to the subject. In still another embodiment, the step of assessing also includes inducing hypertrophy in the subject prior to administering the candidate MBL inhibitor. In yet another embodiment, the step of assessing further includes comparing the result of the administration with outcome in a control subject. In a further embodiment, more than one subject is administered the candidate MBL inhibitor and there are more than one control subjects. In one embodiment, the hypertrophy is cardiac hypertrophy. In another embodiment, the hypertrophy is nonischemic.

In another embodiment, the method includes the steps of selecting a candidate MBL inhibitor, and assessing its ability to treat cardiomyopathy in a subject. In yet another embodiment, the step of assessing includes administering the candidate MBL inhibitor to the subject. In a further embodiment, the step of assessing also includes inducing cardiomyopathy in the subject prior to administering the candidate MBL inhibitor. In yet a further embodiment, the step of assessing further includes comparing the result of the administration with outcome in a control subject. In another embodiment, the cardiomyopathy is nonischemic cardiomyopathy. In a further embodiment, more than one subject is administered the candidate MBL inhibitor and there are more than one control subjects.

In yet another embodiment, the method includes the steps of selecting a candidate MBL inhibitor, contacting the candidate MBL inhibitor with cardiac cells, and assessing its ability to inhibit hyperglycemic myocardial damage. In one embodiment, the contacting is carried out by administering the candidate MBL inhibitor to a subject. In another embodiment, the step of assessing also includes inducing hyperglycemia in the subject prior to administering the candidate MBL inhibitor. In yet another embodiment, the step of assessing also includes inducing diabetes in the subject prior to administering the candidate MBL inhibitor. In a further embodiment, the step of assessing further includes comparing the result of the administration with outcome in a control subject. In yet a further embodiment, more than one subject is administered the candidate MBL inhibitor and there are more than one control subjects. In one embodiment, the contacting is carried our in vitro. In another embodiment, the cardiac cells are cardiac fibroblasts, endothelial cells, mast cells or vascular smooth muscle cells.

In still another embodiment, the method includes the steps of selecting a candidate MBL inhibitor, contacting the candidate MBL inhibitor with cardiac cells, and assessing its ability to inhibit cardiac progenitor cell loss. In one embodiment, the contacting is carried out by administering the candidate MBL inhibitor to a subject. In another embodiment, the step of assessing also includes inducing cardiac progenitor cell loss in the subject prior to administering the candidate MBL inhibitor. In one embodiment, the step of assessing also includes inducing hyperglycemia in the subject prior to administering the candidate MBL inhibitor. In another embodiment, the step of assessing also includes inducing diabetes in the subject prior to administering the candidate MBL inhibitor. In a further embodiment, the step of assessing further includes comparing the result of the administration with outcome in a control subject. In one embodiment, more than one subject is administered the candidate MBL inhibitor and there are more than one control subjects. In another embodiment, the contacting is carried our in vitro. In yet another embodiment, the cardiac cells are cardiac fibroblasts, endothelial cells, mast cells or vascular smooth muscle cells.

In a further embodiment, the method includes the steps of selecting a candidate MBL inhibitor, and assessing its ability to inhibit LCP-associated complement activation in a subject with diabetes. In one embodiment, the subject has nonischemic diabetes. In another embodiment, the subject has Type I or Type II diabetes. In yet another embodiment, the subject has diabetic nephropathy. In one embodiment, the step of assessing includes administering the candidate MBL inhibitor to the subject. In another embodiment, the step of assessing also includes inducing diabetes in the subject prior to administering the candidate MBL inhibitor. In a further embodiment, the step of assessing further includes comparing the result of the administration with outcome in a control subject. In one embodiment, more than one subject is administered the candidate MBL inhibitor and there are more than one control subjects.

In yet another aspect, the invention is a method for screening a subject for susceptibility to treatment with a MBL inhibitor. In one embodiment, the method includes the steps of isolating a mammalian cell from a subject, and detecting the presence of a MBL on a surface of the mammalian cell, wherein the presence of the MBL indicates that the cell is susceptible to LCP-associated complement activation and that the subject is susceptible to treatment with a MBL inhibitor. In one embodiment, the method includes the step of contacting the MBL with a detection reagent that selectively binds to the MBL to detect the presence of the MBL. The detection reagent, in one embodiment, is an isolated MBL inhibitor.

A method for screening a subject for susceptibility to treatment with a MBL inhibitor is provided in another aspect. The method includes the steps of contacting a mammalian cell from a subject with a labeled isolated MBL inhibitor or MBL binding peptide, and detecting the presence of a MBL on the surface of the mammalian cell, wherein the presence of the MBL indicates that the cell is susceptible to LCP-associated complement activation and that the subject is susceptible to treatment with a MBL inhibitor. In one embodiment, the mammalian cell is an endothelial cell.

In one embodiment of the aforementioned methods, the subject has hyperglycemia or diabetes. In still another embodiment, the subject has hypertrophy, such as cardiac hypertrophy, or cardiomyopathy. In yet another embodiment, the subject has cardiac progenitor cell loss. In another embodiment, the mammalian cell is a cardiac cell. In a further embodiment, the mammalian cell is a cardiac fibroblast, endothelial cell, mast cell or vascular smooth muscle cell.

In one embodiment of any of the methods provided, the subject is a non-human animal. In another embodiment, the animal is a non-human primate, dog, cat, horse, sheep, goat, cow, rabbit, pig or rodent.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE FIGURES

FIG. 1 illustrates left ventricular function following MI/R. Echocardiography was performed and ejection fraction (%) was calculated from the M-mode measurements. FIG. 1A provides the results from typical M-Mode recordings. In the upper panels: non-diabetic wild-type (WT) mice that were sham-operated or underwent MI/R with 30 or 15 min of ischemia and 4 h reperfusion (I). The lower panels represent diabetic animals after MI/R with 15 min of ischemia (I) and 4 h reperfusion: diabetic WT mice, insulin-treated diabetic WT mice and diabetic MBL A/C KO mice. FIG. 1B provides a summary of ejection fraction data. The left group of bars represents non-diabetic WT mice that were sham-operated (sham) or underwent MI/R with 30 and 15 min of ischemia and 4 h of reperfusion (IR 30 and IR 15, respectively). The right group of bars represents diabetic animals: sham-operated diabetic WT mice (sham), non-treated diabetic WT mice following MI/R (IR 15), insulin-treated diabetic WT mice following MI/R (insulin IR 15) and diabetic MBL A/C KO mice after MI/R with 15 min of ischemia and 4 h of reperfusion (MBL-KO IR 15). All data are mean±SE of 4-6 animals per group. *, p<0.001 compared to non-diabetic WT sham and non-diabetic WT mice following MI/R with 15 min of ischemia (IR 15). **, p<0.001 compared to diabetic WT sham, insulin-treated diabetic WT mice and diabetic MBL A/C KO mice after MI/R with 15 min of ischemia (IR 15). ***, p<0.001 compared to diabetic WT mice following MI/R with 15 min of ischemia (IR 15).

FIG. 2 shows left ventricular area measurements. FIG. 2A provides long axis area measurements. Long axis diastolic (D) and systolic (S) areas (cm2) were measured before MI/R for non-diabetic (WT) mice, diabetic WT (WT diab) mice after 2 weeks of diabetes, insulin treated diabetic WT (WT diab/Ins) mice and diabetic MBL A/C KO (MBLKO diab) mice. All data are mean±SE of 4 animals per group. *, p<0.05 compared to diastolic areas of non-diabetic WT mice. **, p<0.05 compared to diastolic areas of non-treated diabetic WT mice. +, p<0.001 compared to systolic areas of non-diabetic WT mice. ++, p<0.05 compared to systolic areas of non-treated diabetic WT mice. FIG. 2B provides short axis area measurements. Short axis diastolic (D) and systolic (S) areas (cm2) were measured before MI/R for non-diabetic WT (WT) mice, diabetic WT (WT diab) mice after 2 weeks of diabetes, insulin treated diabetic WT (WT diab/Ins) mice and diabetic MBL A/C KO (MBL-KO diab) mice. All data are mean±SE of 4 animals per group. *, p<0.05 compared to diastolic areas of non-diabetic WT mice. **, p<0.05 compared to diastolic areas of non-treated diabetic WT mice. +, p<0.05 compared to systolic areas of non-diabetic WT mice. ++, p<0.05 compared to systolic areas of non-treated diabetic WT mice.

FIG. 3 shows heart/body weight ratios. In some animals that did not undergo MI/R, heart and body weight values were obtained in non-diabetic and diabetic animals at 10 weeks of age. In diabetic animals, diabetes was induced at 8 weeks of age. Heart/body weight ratios were determined. All data are mean±SE of 4 animals per group. *, p<0.05 compared to non-diabetic WT mice. **, p<0.05 compared to diabetic WT mice. Diab/ins=diabetes and insulin treated; MBL-KO diab=MBL null mice made diabetic.

FIG. 4 provides serum troponin I concentrations after MI/R. Serum troponin I concentrations after MI/R were measured. The left group of bars represents non-diabetic WT mice that underwent MI/R with 15 or 30 min of ischemia and 4 h reperfusion (IR). The right group of bars represents diabetic animals that underwent 15 min of ischemia and 4 h of reperfusion (IR): nontreated diabetic WT mice (IR 15), insulin-treated diabetic WT mice (insulin IR 15) and nontreated diabetic MBL A/C KO mice (MBL-KO IR 15). All data are mean±SE of 4-6 animals per group. p<0.05 compared to non-diabetic WT mice following MI/R with 15 min of ischemia. **, p<0.05 compared to diabetic WT mice following MI/R with 15 min of ischemia.

FIG. 5 illustrates neutrophil infiltration into the myocardium following MI/R. Heart sections were stained and neutrophil infiltration into the myocardium was measured using an infrared imaging system (LICOR). Representative sections are shown from 3 animals per group. Upper row: 1.+2. Ab (primary and secondary antibody). Lower row: control group with 2. Ab only (secondary only antibody). Groups: non-diabetic WT mice following MI/R with 30 min of ischemia (I) and 15 min of ischemia, respectively; diabetic WT mice following MI/R with 15 min of ischemia untreated and insulin-treated, respectively; diabetic MBL A/C KO mice following MI/R with 15 min of ischemia.

FIG. 6 provides MBL levels in patients with acute MI. Patients with acute MI from TIMI 14 and ENTIRETIMI 23 trials were analyzed for MBL concentrations. Diabetic status was then evaluated and the patients broken into diabetic and non-diabetic groups. All data are mean±SE of 643 patients in the non-diabetic group and 104 patients in the diabetic group. *, p<0.05 compared to non-diabetic patients with acute MI.

FIG. 7 provides cardiac progenitor cell (CPC) numbers. Heart sections were stained and CPC amounts were measured using an infrared imaging system (LI-COR). Representative sections are shown from 3 animals per group. Upper row: 1.+2. Ab (primary and secondary antibody). Lower row: control group with 2. Ab only (secondary only antibody). Groups: nondiabetic WT mice; diabetic WT mice following two weeks of diabetes untreated and insulin treated, respectively; diabetic MBL A/C KO mice following 2 weeks of diabetes.

### BRIEF DESCRIPTION OF THE INVENTION

Chronic hyperglycemia has adverse effects on the myocardium, for example through microvascular impairment and changes in myocardial metabolism, and there is increasing evidence that acute hyperglycemia is detrimental in the setting of myocardial infarction. Animal studies indicate that hyperglycemia adversely affects cardiovascular responses to ischemia. For example, acute hyperglycemia impairs endothelial function, attenuates coronary microcirculatory responses to myocardial ischemia, and markedly attenuates cardioprotective signal transduction. Studies show that myocardial infarct size is directly related to blood glucose concentration and that acute hyperglycemia abolishes infarct size reduction in response to ischemic or pharmacological preconditioning. Hyperglycemia also increases the production of reactive oxygen species, induces oxidative stress, and attenuates nitric oxide signaling. In the clinical arena, hyperglycemia is an independent predictor of cardiovascular morbidity and mortality. Results from a large nationwide French registry indicate that hyperglycemia is associated with poor outcomes in non-diabetics with acute myocardial infarction. It has also been found that elevated fasting glucose levels are an important independent risk factor for 30-day mortality in patients with acute myocardial infarction and that stress hyperglycemia, commonly found among patients with acute myocardial infarction, is associated with an increased risk of in-hospital mortality in patients with and without diabetes. A recent meta-analysis reported that up to 71% of non-diabetics admitted to hospital with acute myocardial infarction had stress hyperglycemia, while for diabetics the values ranged from 46% to 84%. Hyperglycemia, such as acute hyperglycemia, is thus a common problem in both recognized diabetic and non-diabetic populations and appears to contribute to cardiovascular damage.

The complement system plays a role in myocardial ischemia and reperfusion (MI/R) injury¹⁻³ as well as ischemia and reperfusion (I/R) injury of other organs, such as muscle, ileum and kidney⁴⁻⁶. In diabetes, I/R injury is often enhanced. Studies have shown elevated MI/R injury in diabetic animals¹³ and suggested a role for reactive oxygen and nitrogen species^{14,15}. These laboratory observations mirror data from clinical studies. Diabetic patients have increased incidences of atherosclerosis, including coronary artery disease and myocardial infarction (MI)^{16,17}. Diabetics also have increased mortality and worse long-term prognosis after MI compared to non-diabetic patients¹⁷⁻¹⁹. While studies have demonstrated the role of the complement system and MBL in MI/R injury^{1,2,10}, a full understanding of complement and MBL in increasing injury in diabetics compared to non-diabetics, including injury without I/R, has not been previously evaluated.

In streptozotocin (STZ)-induced diabetic mice, ejection fraction (EF) was significantly decreased and troponin I levels and myocardial neutrophil infiltration significantly increased compared to non-diabetic mice following MI/R. Diabetic MBL-deficient mice were significantly protected from MI/R injury compared to diabetic wild type (WT) mice. Echocardiographic measurements in diabetic mice demonstrated signs of dilative cardiomyopathy and heart/body weight ratios suggested hypertrophic cardiac remodeling. Interestingly, it was also found that the absence of MBL inhibited myocardial damage in hyperglycemic subjects in the absence of I/R and increased injury as a result of I/R. Further, immunohistochemical analysis of cardiac progenitor cells (CPC) revealed significantly lower numbers in diabetic WT hearts compared to non-diabetics. Insulin-treated diabetic WT or untreated diabetic MBL-deficient mice were protected from dilative cardiomyopathy, hypertrophic remodeling and loss of cardiac CPC.

Therefore, in one aspect, methods and compositions for inhibiting hyperglycemic myocardial damage are provided. Such a method, in one embodiment, can include the step of contacting a cardiac cell or cardiac cells with an effective amount of a MBL inhibitor to inhibit LCP-associated complement activation. The effective amount for such a method is also one that is effective in inhibiting hyperglycemic myocardial damage. The method, in another embodiment, can include the step of administering a MBL inhibitor to a subject in need thereof. As used herein, “a subject in need thereof” in reference to this method is one in which the inhibition of hyperglycemic myocardial damage would be of some benefit.

As used herein, “hyperglycemic myocardial damage” refers to any injury to cardiac tissue that is the result of hyperglycemia (i.e., the condition of higher than normal blood sugar) and includes cardiac hypertrophy and cardiomyopathy. One of ordinary skill in the art is familiar with methods for determining the level of blood sugar in a subject as well as determining whether the level is higher than normal. One of ordinary skill in the art, therefore, is familiar with methods to determine whether or not a subject is hyperglycemic. Therefore, in some embodiments, the methods provided herein include the step of assessing the blood sugar level in the subject prior to the administration of the MBL inhibitor. In one embodiment, therefore, the subject that is administered the MBL inhibitor has a higher than normal level of blood sugar. The hyperglycemia of the subject can be chronic hyperglycemia or the hyperglycemia can be acute hyperglycemia, such as stress hyperglycemia. In some embodiments, the subject is hyperglycemic but would not be considered to have diabetes by a clinician. In other embodiments, the subject is hyperglycemic and would be considered to have diabetes by a clinician. The term “diabetes”, as used herein, includes Type I and Type II diabetes. A “diabetic subject”, as used herein, is one that has or would be diagnosed to have diabetes by an ordinarily skilled clinician. In another embodiment, the subject is MBL sufficient. As used herein, “MBL sufficient” refers to a subject in which MBL is present and the administration of a MBL inhibitor would result in the inhibition of LCP-associated complement activation. In one embodiment, this inhibition is measurable. In another embodiment, this inhibition is measurable and statistically significant. The method, in some embodiments, therefore, can also include a step of determining whether or not the subject is MBL sufficient. One of ordinary skill in the art is familiar with methods for detecting MBL. Additionally, methods for detecting MBL are provided elsewhere herein. Further, methods can also be found in U.S. Pat. No. 7,273,925. Such methods are incorporated herein by reference in their entirety.

The cardiac cell can be any cardiac cell in which the cell surface carbohydrates or peptides interact with MBL. Cardiac cells include cardiac fibroblasts, cardiac endothelial cells, cardiac mast cells and cardiac vascular smooth muscle cells. Cardiac cells having MBL ligands can easily be identified as provided above and elsewhere herein. For instance, a MBL binding assay can be used to identify MBL ligands. In some embodiments, a number of cardiac cells are contacted with the MBL inhibitor. Therefore, the method can also be one in which cardiac tissue is contacted with a MBL inhibitor. In one embodiment, the contacting is carried out in vitro. In some embodiments, the method can further include the step of obtaining a cardiac cell or cells or cardiac tissue from the subject prior to contact with the MBL inhibitor.

In another aspect, methods and compositions for regulating lectin complement pathway (LCP)-associated complement activation in subjects with cardiomyopathy and/or hypertrophy are provided. In yet another aspect, methods and compositions for regulating lectin complement pathway (LCP)-associated complement activation to inhibit the loss of cardiac progenitor cells are provided.

In one aspect, a method for inhibiting LCP-associated complement activation in a subject with hypertrophy is provided. In one embodiment, the method can include the step of contacting a mammalian cell having surface exposed MBL ligand with an effective amount of a MBL inhibitor to inhibit LCP-associated complement activation. The effective amount, in some embodiments, is an amount effective to treat the hypertrophy in the subject. The step can be accomplished by administering an effective amount of a MBL inhibitor to inhibit LCP-associated complement activation in the subject.

As used herein, “hypertrophy” refers to an increase in size of an organ or tissue. The hypertrophy can be cardiac hypertrophy (e.g., left ventricle hypertrophy) but is not so limited. The hypertrophy can, in some embodiments, be nonischemic hypertrophy. “Nonischemic” refers to hypertrophy that occurs independent of (i.e., without) I/R. Nonischemic hypertrophy can, in some embodiments, occur in addition to ischemic hypertrophy. In some embodiments, the subject is hyperglycemic. In other embodiments, the subject has chronic hyperglycemia or acute hyperglycemia. In some embodiments, the method can also include the step of assessing the blood sugar level in the subject prior to the administration of the MBL inhibitor. In one embodiment, therefore, the subject that is administered the MBL inhibitor has a higher than normal level of blood sugar. In some embodiments, the subject is hyperglycemic but would not be considered to have diabetes by a clinician. In other embodiments, the subject is hyperglycemic and would be considered to have diabetes by a clinician. In some embodiments, the diabetes is Type I or Type II diabetes. In another embodiment, the subject is MBL sufficient. The method, in some embodiments, can also include a step of determining whether or not the subject is MBL sufficient.

The mammalian cell can be any cell in which the cell surface carbohydrates or peptides interact with MBL. In one embodiment, the mammalian cell is an endothelial cell having a surface exposed MBL ligand. In another embodiment, the mammalian cell is a cardiac cell. Mammalian cells include cardiac fibroblasts, endothelial cells, mast cells and vascular smooth muscle cells. Mammalian cells having MBL ligands can easily be identified as described elsewhere herein.

In yet another aspect, a method for inhibiting LCP-associated complement activation in a subject with cardiomyopathy is also provided. The method can include the step of contacting a mammalian cell, such as a cardiac cell, having surface exposed MBL ligand with an effective amount of a MBL inhibitor to inhibit LCP-associated complement activation. The effective amount, in some embodiments, is an amount effective to treat the cardiomyopathy in the subject. The step can be accomplished by administering an effective amount of a MBL inhibitor to inhibit LCP-associated complement activation in the subject.

As used herein, “cardiomyopathy” refers to a weakening of the heart muscle or a change in heart muscle structure often associated with inadequate heart pumping or other heart function abnormalities. The cardiomyopathy, in some embodiments, is nonischemic cardiomyopathy (i.e., cardiomyopathy that is independent of I/R) and can be in addition to ischemic cardiomyopathy. Nonischemic cardiomyopathy includes dilated cardiomyopathy, hypertrophic cardiomyopathy, arrhythmogenic right ventricular cardiomyopathy or restrictive cardiomyopathy. In some embodiments, the subject is hyperglycemic. In other embodiments, the subject has chronic hyperglycemia or acute hyperglycemia. In some embodiments, the method can also include the step of assessing the blood sugar level in the subject prior to the administration of the MBL inhibitor. In one embodiment, therefore, the subject that is administered the MBL inhibitor has a higher than normal level of blood sugar. In some embodiments, the subject is hyperglycemic but would not be considered to have diabetes by a clinician. In other embodiments, the subject is hyperglycemic and would be considered to have diabetes by a clinician. In some embodiments, the diabetes is Type I or Type II diabetes. In another embodiment, the subject is MBL sufficient. The method, in some embodiments, can also include a step of determining whether or not the subject is MBL sufficient.

In a further aspect, a method for inhibiting the loss of cardiac progenitor cells is provided. Such a method, in one embodiment, can include the step of contacting a cardiac cell or cardiac cells with an effective amount of a MBL inhibitor to inhibit LCP-associated complement activation. The effective amount for such a method is also one that is effective in inhibiting cardiac progenitor cell loss. The method, in another embodiment, can include the step of administering a MBL inhibitor to a subject in need thereof. As used herein, “a subject in need thereof” in reference to this method is one in which the inhibition of cardiac progenitor cell loss would be of some benefit.

Also, as used herein, “loss of cardiac progenitor cells” refers to any reduction in the number of cardiac progenitor cells that would occur in the absence of the MBL inhibitor as compared to the reduction in the number of cardiac progenitor cells in the presence of the MBL inhibitor. Loss of cardiac progenitor cells can be induced by hyperglycemia. As used herein, “cardiac progenitor cells” refers to cells that can proliferate and differentiate into cardiomyocytes, vascular smooth muscle cells or endothelial cells.

In some embodiments, the method includes the step of assessing the blood sugar level in the subject prior to the administration of the MBL inhibitor. In one embodiment, therefore, the subject that is administered the MBL inhibitor has a higher than normal level of blood sugar. The hyperglycemia of the subject can be chronic hyperglycemia or the hyperglycemia can be acute hyperglycemia. In some embodiments, the subject is hyperglycemic but would not be considered to have diabetes by a clinician. In other embodiments, the subject is hyperglycemic and would be considered to have diabetes by a clinician. In some embodiments, the diabetes is Type I or Type II diabetes. In another embodiment, the subject is MBL sufficient. The method, in some embodiments, can also include a step of determining whether or not the subject is MBL sufficient.

In yet a further aspect, a method for inhibiting LCP-associated complement activation in a subject with diabetes is provided. In one embodiment, the method can include the step of contacting a mammalian cell having surface exposed MBL ligand with an effective amount of a MBL inhibitor to inhibit LCP-associated complement activation. The effective amount, in some embodiments, is an amount effective to treat the diabetes in the subject. In other embodiments, this amount is effective to inhibit hyperglycemic myocardial damage in the subject. This step can be accomplished by administering an effective amount of a MBL inhibitor to inhibit LCP-associated complement activation in the subject. The diabetic subject can be a nonischemic diabetic subject. In some embodiments, the subject has Type I of Type II diabetes. In some embodiments, the method includes the step of assessing the blood sugar level in the subject prior to the administration of the MBL inhibitor. In one embodiment, therefore, the subject that is administered the MBL inhibitor has a higher than normal level of blood sugar. In another embodiment, the subject is MBL sufficient. The method, in some embodiments, can also include a step of determining whether or not the subject is MBL sufficient.

A “nonischemic diabetic subject”, as used herein, is one with diabetes and in which I/R or tissue injury as a result of I/R is not at present occurring and/or has not occurred. In some embodiments, the subject has diabetic nephropathy. The diabetic nephropathy, in some embodiments, is not the result of I/R injury.

As used herein, the subject of any of the methods, in some embodiments, is one in which I/R is not at present occurring as determined by an ordinarily skilled clinician. In other embodiments, the subject is one in which I/R is not occurring and has not occurred. The subject, therefore, in some embodiments, is one in which treatment with a MBL inhibitor to treat or prevent I/R tissue injury would not ordinarily be prescribed. The subject can also, in other embodiments, be one that does not have and/or would not be diagnosed to have atherosclerosis, arthritis, myocardial infarction, transplantation, CPB, stroke, ARDS, SLE, Lupus or dialysis. The subject, in still other embodiments, is one that does not have and/or would not be diagnosed to have hypertension or other cardiac disease. Each of these disorders is well-known in the art and is described, for instance, in Harrison's Principles of Internal Medicine (McGraw Hill, Inc., New York). In still further embodiments, the subject is one that does not have and/or would not be diagnosed to have a disease, disorder or condition that would lead to I/R or tissue injury as a result of I/R. In further embodiments, the subject would not be a subject as defined in U.S. Pat. No. 7,273,925. The description of such subjects is incorporated herein by reference in its entirety; in some embodiments, such subjects are excluded from the subjects provided herein.

The methods provided can be carried out in vivo or in vitro. The step of “contacting”, as used herein, refers to the addition of the MBL inhibitor to a medium containing a mammalian cell. The medium can be an in vitro tissue culture or a biological specimen, an ex vivo sample, or in vivo. The step of contacting refers to the addition of the MBL inhibitor in such a manner that it will prevent LCP-associated complement activation associated with the mammalian cell and/or effect the desired therapeutic endpoint.

An “MBL inhibitor” as used herein is a compound that prevents LCP-associated complement activation. The MBL inhibitor, for example, can be a molecule that binds any molecule involved in LCP-associated complement activation. The MBL inhibitor can function, as another example, by blocking MBL deposition on the surface of a mammalian cell or by blocking the association of MASP-1 or MASP-2 or C3b associated with MBL deposition.

The MBL inhibitor can be, for example, an isolated MBL binding peptide. An “isolated MBL binding peptide”, as used herein, is a peptide which binds to MBL and inhibits LCP-associated complement activation. One method by which MBL binding peptides inhibit LCP-associated complement activation is by binding to MBL and inhibiting MBL association with surface exposed MBL ligands. Additionally, the MBL binding peptide may bind to MBL and inhibit the association between MBL and MASP-1 or -2 and/or C3b. Several peptides which bind to MBL or MASP have been described in the art, including Lanzrein, A. S. et al., Department of Pharmacology, University of Oxford, UK, May 11, 1998, Neuroreport, 9(7):1491-5; Jack, D. L. et al., Immunobiology Unit, Institute of Child Health, London, UK, J Immunol, Feb. 1, 1998, 160(3):1346-53, Terai, I. et al., Division of Clinical Pathology, Hokkaido Institute of Public Health, Sapporo, Japan, Clin. Exp. Immunol., November, 1997, 110(2):317-23; Endo, M. et al., Second Department of Internal Medicine, Nihon University School of Medicine, Tokyo, Japan, Nephrol Dial Transplant, Aug. 13, 1998, (8):1984-90; Valdimarsson, H. et al., Department of Immunology, University of Reykjavik, Iceland, Scand. J. Immunol., August 1998, 48(2):116-23; Thiel, S. et al., Clin Exp. Immunol., October 1992, 90(1):31-5.

The MBL binding peptide can be one that selectively binds to a human MBL epitope and that inhibits LCP-associated complement activation. A “human MBL epitope”, as used herein, is a portion of MBL which, when contacted with a MBL-binding peptide, inhibits LCP-associated complement activation by preventing the association between MBL and the MBL ligand or MASP-1 or -2 and/or C3b. In some embodiments, the MBL epitope can be a region of the MBL which interacts with any of the monoclonal antibodies produced by the hybridomas deposited with the ATCC under ATCC Accession No. (HB-12621), ATCC Accession No. (HB-12620) and ATCC Accession No. (HB-12619). The MBL binding peptide, therefore, can be a monoclonal antibody, such as one of the aforementioned deposited antibodies.

The MBL inhibitor can also be an isolated MASP binding peptide. An “isolated MASP binding peptide”, as used herein, refers to a peptide which binds to MASP-1 and/or MASP-2 and prevents LCP-associated complement activation by preventing MASP-1 and/or MASP-2 from forming a complex with MBL on the surface of a cell thereby preventing the resulting C3b deposition associated with the MBL-MASP complex. In some embodiments, the MBL inhibitor is a MASP-2 inhibitor. In some embodiments, the MASP-2 inhibitor is a C1 inhibitor. MASP-2 inhibitors have been reported to include FUT175 as well as the inhibitors of application Ser. Nos. 11/150,883 and 11/645,359. MASP-2 inhibitors also include antibodies to MASP-2. MASP-2 antibodies have been reported to include the antibodies of application Ser. No. 10/556,509. The description of these reported inhibitors is incorporated herein by reference.

The MBL inhibitor can also be a mannose (mannan) binding peptide. A “mannose (mannan) binding peptide”, as used herein, is a peptide which binds to the MBL ligand on the surface of a mammalian cell, preventing its interaction with the MBL-MASP complex. MBL inhibitors also include the MBL inhibitors of U.S. Pat. No. 7,273,925 and the MBL receptor antagonists as described in copending application Ser. No. 09/638,420. The MBL inhibitors of this patent and application are incorporated herein by reference in their entirety. As used herein, a “MBL receptor antagonist” is a compound that prevents LCP-associated complement activation by inhibiting MBL binding to a MBL ligand (alternatively referred to as an “MBL receptor”) expressed on the surface of a mammalian cell.

A MBL inhibitor can be a plant lectin or a functional equivalent peptide derived therefrom. The MBL inhibitor can be a legume-derived lectin or a functional equivalent thereof. A legume-derived lectin can be an isolated peptide (naturally occurring or synthetic) derived from a legume. The legume derived lectin, in some embodiments, is Ulex europaeus (UEA)-II, Laburnum alpinum (LAA)-I, or Cytisus Sessilifolius anti-H(O) Lectin 1 (CSA-1). A functional equivalent of a legume derived lectin is a molecule, peptide or non-peptide, which has an equivalent function to the legume derived lectin, such as a molecule having conservative substitutions.

As used herein, “conservative substitution” refers to an amino acid substitution which does not alter the relative charge or size characteristics of the peptide in which the amino acid substitution is made. Conservative substitutions of amino acids include substitutions made amongst amino acids with the following groups: (1) M,I,L,V; (2) F,Y,W; (3) K,R,H; (4) A,G; (5) S,T; (6) Q,N; and, (7) E,D. Such substitutions can be made by a variety of methods known to one of ordinary skill in the art. For example, amino-acid substitutions may be made by PCR-directed mutation, site-directed mutagenesis according to the method of Kunkel (Kunkel, Proc. Nat. Acad. Sci. U.S.A. 82: 488-492, 1985) or by chemical synthesis. These and other methods are known to those of ordinary skill in the art and may be found in references which compile such methods, e.g. Sambrook. et al., Molecular Cloning: A Laboratory Manual, 2nd edition, Cold Spring Harbor Laboratory Press, 1989. The activity of functionally equivalent variants of MBL inhibitors can be tested by the binding and activity assays discussed herein or otherwise known to those of ordinary skill in the art.

The MBL inhibitor can also be a keratin binding molecule, such as a keratin antibody. Many keratin antibodies are commercially available. These include, but are not limited to, those antibodies commercially available from Research Diagnostics, Inc., e.g., RDI-CYTOK1abr (Cytokeratin 1 rabbit polyclonal); RDI—(mouse cytokeratins rabbit polyclonals); RDI-CBL222 (Cytokeratin 1-3 AE8+mIgG1); RDI-PRO61808 (Cytokeratin 1,10/11 K8.60+mIgG1); RDI-PRO65177 (Cytokeratin 2E Ks 2.398.3.1 mIgG1); RDI-PRO65191 (Cytokeratin 2E Ks2.342.7.1+mIgG1); RDI-CBL218 (Cytokeratin 3 (bovine & rabbit) AE5 mIgG1); RDI-PRO10525 (Cytokeratin 4 (most mammals) 6B10 mIgG1); RDI-CBL234 (Cytokeratin 4,5,6,8,10,13,18 C11 mIgG); RDI-CBL232 (Cytokeratin 5,8 C50+mIgG1); RDI-PRO10521 (Cytokeratin 5+8 RCK102+mIgG1); RDI-PRO61531 (Cytokeratin 5+8 RCK12+Biotin conj); RDI-PRO61431 (Cytokeratin 5+8 RCK102+FITC conj); RDI-PRO61031 (Cytokeratin 5+8(pan epithelial) C22 mIgG1); RDI-PRO65190 (Cytokeratin 6 Ks6.Ka12+mIgG1); RDI-CBL194 (Cytokeratin 7 LP5K mIgG2b); RDI-CBL184 (Cytokeratin 7 Lds68 IgM); RDI-PRO61025 (Cytokeratin 7 (bovine, sheep, pig) KS7.18+mIgG1); RDI-PRO10522 (Cytokeratin 7 (hamster, mouse) RCK105 mIgG1); RDI-CBL195 (Cytokeratin 8 LP3K mIgG1); RDI-CBL195FT (Cytokeratin 8 LP3K mIgG1 FITC); RDI-PRO61038 (Cytokeratin 8 (mouse, rat, pig, hamster) Ks8.7+*mIgG1); RDI-PRO65130 (Cytokeratin 8(phosphorylated) Ks 8-17.2 mIgG1); RDI-CBL170 (Cytokeratin 8,18 5D3+mIgG2a); RDI-PRO10526 (Cytokeratin 8 (most mammals) M20 mIgG1); RDI-PRO651104 (Cytokeratin 9 KS9.7 & KS 9.216 mIgG1/mIgG3); RDI-CBL196 (Cytokeratin 10 LH2 mIgG); RDI-PRO10501 (Cytokeratin 10 (rat, mouse, bovine, rabbit) RKSE60 mIgG1); RDI-PRO11414 (Cytokeratin 10 DE-K10+mIgG1); RDI-CBL217 (Cytokeratin 10,11,1 & 2 AE2+mIgG1); RDI-PRO61007 (Cytokeratin 13 (bovine & rat) Ks13.1+mIgG1); RDI-PRO10523 (Cytokeratin 13 (human, mouse, rabbit) 1C7 mIgG2a); RDI-PRO10524 (Cytokeratin 13 (human, mouse, rabbit) 2D7 mIgG2b); RDI-CBL197 (Cytokeratin 14 LL002+mIgG3); RDI-CBL197FT (Cytokeratin 14 LL002+mIgG3 FITC); RDI-PRO10003 (Cytokeratin 14 RCK 107 mIgG1); RDI-PRO61036 (Cytokeratin 17 (rat 46 kDa polyp) KS17.E3+mIgG2b); RDI-CBL236 (Cytokeratin 18 CO4+mIgG1); RDI-PRO61028 (Cytokeratin 18 (mouse, rat, pig, dog, sheep, hamster, bovine, trout) Ks18.04+mIgG1); ARDI-PRO11416 (Cytokeratin 18 RCK 106 mIgG1); RDI-PRO10500 (Cytokeratin 18 RGE 53 mIgG1); RDI-CBL185 (Cytokeratin 18 DC10+mIgG1); RDI-CBL178 (Cytokeratin 19 Ks19.1+mIgG2a); RDI-CBL198 (Cytokeratin 19 BA17+mIgG1); RDI-PRO61029 (Cytokeratin 19 (rat, fish, bovine) KS19.2 mIgG2b); RDI-CBL199 (Cytokeratin 19 LAS86 mIgM); RDI-CBL247 (Cytokeratin 19 A53-B/A2.26 mIgG2a); RDI-CBL208 (Cytokeratin 20 Ks20.8+mIgG1); RDI-PRO61054 (Cytokeratin 20 (rat, pig) mouse IT-KS20.10+mIgG1); RDI-PRO61033 (Cytokeratin 20 Ks20.5 mIgG2a); RDI-CBL215 (Cytokeratin Type I (monkey, rabbit, mouse, rat, bovine, chicken, monkey) AE1+mIgG1); RDI-CBL216 (Cytokeratin Type II monkey, rabbit, mouse, rat, bovine, chicken, monkey) AE3+mIgG1); RDI-PRO61031 (Cytokeratin (Pan epithelial) C22 mIgG1); RDI-PRO61006 (Cytokeratin TYPE II Ks pan 1-8 mIgG2a); RDI-PRO61056 (Cytokeratin TYPE II “ ” Biotin labeled); RDI-PRO61406 (Cytokeratin Type II “ ” FITC conjugated); and RDI-PRO61835 (Cytokeratin Type I & II AE1/AE3 mIgG1). These antibodies also include anti-pan-cytokeratin (human, bovine rat and mouse, catalog number 250400) from CALBIOCHEM, and product numbers C7034 (anti-cytokeratin 8.12); C6909 (anti-cytokeratin 8.13); C7284 (anti-cytokeratin 8.60); C7785 (anti-cytokeratin CK5); C8791 (anti-cytokeratin peptide 14); and C1399 (anti-cytokeratin peptide 18) from Sigma-Aldrich.

In some embodiments, the MBL inhibitor is INSP052 of WO2006/120160. In other embodiments, the MBL inhibitor is not INSP052. In still other embodiments, the MBL inhibitor is a siRNA molecule. As used herein, a “siRNA molecule” is a double stranded RNA molecule (dsRNA) consisting of a sense and an antisense strand, which are complementary (Tuschl, T. et al., 1999, Genes & Dev., 13:3191-3197; Elbashir, S. M. et al., 2001, EMBO J., 20:6877-6888). Inhibitory siRNA molecules have been reported to include those of application Ser. No. 11/615,554. The description of such reported siRNA molecules is incorporated herein by reference. In a further embodiment, the MBL inhibitor is an aptamer (i.e., short DNA or RNA that binds to a target molecule, in this case any molecule involved in lectin complement pathway-associated complement activation). Examples of complement binding aptamers and methods of their production are reported in WO2007/103549. The description of such aptamers and methods of their production, as they pertain to the inhibition of LCP-associated complement activation, is incorporated herein by reference.

Other MBL inhibitors can be identified using routine assays, such as with binding, competition assays and LCP complement activation assays. For example, the ability of a MBL inhibitor to block MBL deposition or prevent association of MASP-1, MASP-2 or C3b with MBL can be detected using routine in vitro binding assays, such as the following assay.

MBL deposition (or association with MASP-1, MASP-2, or C3b) can be measured by ELISA on normoxic HUVECs and HUVECs subjected to 24 hr of hypoxia followed by 3 hr of reoxygenation in the presence of 30% human serum (HS) or 30% HS treated with 3, 30, or 300 mmol/L of N-acetyl-D-glucosamine (GluNAc) or with the putative binding peptide to inhibit competitively MBL deposition. C3 and MBL specific cell surface ELISAs can be performed using peroxidase-conjugated polyclonal goat anti-human C3 antibody (Cappel, West Chester, Pa.) and monoclonal anti-human MBL antibody (Biodesign, Kennebunk, M E, clone #131-1), respectively. HUVECs are grown to confluence on 0.1% gelatinized 96-well plastic plates (Corning Costar, Cambridge, Mass.). The plates are then subjected to 0 (normoxia) or 24 hr of hypoxia. Hypoxic stress is maintained using a humidified sealed chamber (Coy Laboratory Products, Inc., Grass Lake, Mich.) at 37° C. gassed with 1% O₂, 5% CO₂, balance N₂ (Collard C D, et al., Circulation, 1997; 96:326-333). Following the specified period of normoxia or hypoxia, the cell media are aspirated and 100 μl of one the following is added to each well: 1) 30% HS, 2) Hank's balanced salt solution, 3) 30% HS+3, 30, or 300 mmol/L GluNAc, 4) 30% HS+3, 30, or 300 mmol/L D-mannose, 5) 30% HS+3, 30, 300 mmol/L L-mannose, 6) 30% MBL-depleted HS 7) 30% MBL-depleted HS+0.6 μg/ml MBL or 8) 30% HS+3, 30, or 300 mmol/L putative MBL binding peptide. Additionally, 100 μl of purified MBL (3-300 ng/ml) is added to select wells to form a standard curve for quantitative analysis of MBL deposition. The cells are then reoxygenated for 3 hr at 37° C. in 95% air and 5% CO₂. The cells are washed and then fixed with 1% paraformaldehyde (Sigma Chemical Co., St. Louis, Mo.) for 30 min. The cells are then washed and incubated at 4° C. for 1.5 hr with 50 μl of peroxidase-conjugated polyclonal goat anti-human C3 antibody (1:1000 dilution) or monoclonal anti-human MBL antibody (1:1000 dilution). The MBL ELISA plates are then washed and incubated for 1 hr at 4° C. with 50 μl of peroxidase-conjugated polyclonal goat anti-mouse IgG secondary antibody (1:1000 dilution). After washing the cells, the plates are developed with 50 μl of ABTS (2,2′-azino-bis(3-ethylbenzthiazoline-6-sulfonic acid)), and read (Molecular Devices, Sunnyvale, Calif.) at 405 nm. Background controls for the C3 ELISA consist of cells to which only the anti-human C3 antibody is added (i.e., no HS) or cells incubated with 30% heat-inactivated HS. Background controls for the MBL ELISA consist of cells to which only secondary antibody and an isotype control monoclonal antibody to porcine C5a are added. Background optical density is subtracted from all groups. ELISA experiments are generally performed 3 times using 6 wells per experimental group. Endothelial C3 and MBL deposition on normoxic vs. hypoxic HUVECs is analyzed by two-way analysis of variance (ANOVA).

Additionally, MBL inhibitors can be identified from other binding methods as well as from combinatorial libraries. Many types of combinatorial libraries have been described. For instance, U.S. Pat. Nos. 5,712,171 (which describes methods for constructing arrays of synthetic molecular constructs by forming a plurality of molecular constructs having the scaffold backbone of the chemical molecule and modifying at least one location on the molecule in a logically-ordered array); 5,962,412 (which describes methods for making polymers having specific physiochemical properties); and 5,962,736 (which describes specific arrayed compounds).

MBL inhibitors can also be identified by methods such as phage display procedures (e.g., methods described in Hart, et al., J. Biol. Chem. 269:12468 (1994)). Hart et al. report a filamentous phage display library for identifying novel peptide ligands for mammalian cell receptors. In general, phage display libraries using, e.g., M13 or fd phage, are prepared using conventional procedures such as those described in the foregoing reference. The libraries display inserts containing from 4 to 80 amino acid residues. The inserts optionally represent a completely degenerate or a biased array of peptides. Ligands that bind selectively are obtained by selecting those phages which express on their surface a ligand that binds to a certain molecule. These phages then are subjected to several cycles of reselection to identify the peptide ligand-expressing phages that have the most useful binding characteristics. Typically, phages that exhibit the best binding characteristics (e.g., highest affinity) are further characterized by nucleic acid analysis to identify the particular amino acid sequences of the peptides expressed on the phage surface and the optimum length of the expressed peptide to achieve optimum binding. Alternatively, such peptide ligands can be selected from combinatorial libraries of peptides containing one or more amino acids. Such libraries can further be synthesized which contain non-peptide synthetic moieties which are less subject to enzymatic degradation compared to their naturally-occurring counterparts.

Additionally, molecular imprinting can be used for the de novo construction of macromolecular structures such as peptides which bind to a particular molecule. See, for example, Kenneth J. Shea, Molecular Imprinting of Synthetic Network Polymers: The De Novo synthesis of Macromolecular Binding and Catalytic Sites, TRIP Vol. 2, No. 5, May 1994; Klaus Mosbach, Molecular Imprinting, Trends in Biochem. Sci., 19(9) January 1994; and Wulff, G., in Polymeric Reagents and Catalysts (Ford, W. T., Ed.) ACS Symposium Series No. 308, pp 186-230, American Chemical Society (1986). One method for preparing mimics of MBL inhibitors involves the steps of: (i) polymerization of functional monomers around a known MBL inhibitor that exhibits a desired activity; (ii) removal of the template molecule; and then (iii) polymerization of a second class of monomers in the void left by the template, to provide a new molecule which exhibits one or more desired properties which are similar to that of the template. In addition to preparing peptides in this manner other MBL binding molecules which are MBL inhibitors such as polysaccharides, nucleosides, drugs, nucleoproteins, lipoproteins, carbohydrates, glycoproteins, steroids, lipids, and other biologically active materials can also be prepared. This method is useful for designing a wide variety of biological mimics that are more stable than their natural counterparts, because they are typically prepared by the free radical polymerization of functional monomers, resulting in a compound with a nonbiodegradable backbone. Other methods for designing such molecules include for example drug design based on structure activity relationships which require the synthesis and evaluation of a number of compounds and molecular modeling.

By using known monoclonal antibodies, it is also possible to produce anti-idiotypic antibodies which can be used to screen other antibodies to identify whether the antibody has the same binding specificity as the known monoclonal antibody. Such anti-idiotypic antibodies can be produced using well-known hybridoma techniques (Kohler and Milstein, Nature, 256:495, 1975). An anti-idiotypic antibody is an antibody which recognizes unique determinants present on the known monoclonal antibodies. These determinants are located in the hypervariable region of the antibody. It is this region which binds to a given epitope and, thus, is responsible for the specificity of the antibody. An anti-idiotypic antibody can be prepared by immunizing an animal with the known monoclonal antibodies. The immunized animal will recognize and respond to the idiotypic determinants of the immunizing known monoclonal antibodies and produce an antibody to these idiotypic determinants. By using the anti-idiotypic antibodies of the immunized animal, which are specific for the known monoclonal antibodies, it is possible to identify other clones with the same idiotype as the known monoclonal antibody used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas to expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody.

Further, a template, such as a monoclonal antibody can be used to identify other antibodies that bind the same target and inhibit LCP-associated complement activation. It is now routine to produce large numbers of molecules having inhibitory functions based on one or a few peptide sequences or sequence motifs. (See, e.g., Bromme, et al., Biochem. J. 315:85-89 (1996); Palmer, et al., J. Med. Chem. 38:3193-3196 (1995)). Such methods are useful for designing a wide variety of biological mimics that are more stable than the natural counterpart. The created molecules could have the same binding properties as the antibody but be more stable in vivo. Other methods for designing such molecules include, for example, drug design based on structure activity relationships which require the synthesis and evaluation of a number of compounds and molecular modeling.

Whether a particular compound can inhibit LCP-associated complement activation can be assessed using routine in vitro screening assays. For instance, the complement hemolytic assay (CH₅₀) can be performed on MBL-depleted HS in order to demonstrate that MBL depletion inhibits LCP-associated complement activation. The assay may be performed, however, using MBL containing HS and adding a MBL inhibitor and/or a control.

Activation assays also can be used to assess the relative inhibitory concentrations of a MBL inhibitor and to identify those MBL inhibitors which inhibit activation by at least, e.g., 75%.

Other assays will be apparent to those of skill in the art, having read the present specification, which are useful for determining whether a MBL inhibitor inhibits LCP-associated complement activation.

The peptides can be isolated peptides. As used herein, with respect to peptides, the term “isolated peptides” means that the peptides are substantially pure and are essentially free of other substances with which they may be found in nature or in vivo systems to an extent practical and appropriate for their intended use. In particular, the peptides are sufficiently pure and are sufficiently free from other biological constituents of their hosts cells so as to be useful in, for example, producing pharmaceutical preparations or sequencing. Because an isolated peptide of the invention may be admixed with a pharmaceutically acceptable carrier in a pharmaceutical preparation, the peptide may comprise only a small percentage by weight of the preparation. The peptide is nonetheless substantially pure in that it has been substantially separated from the substances with which it may be associated in living systems.

In one embodiment the peptide that inhibits the activation of LCP-associated complement is an antibody or an antigen-binding fragment thereof. Antigen-binding fragments are well known in the art and are regularly employed both in vitro and in vivo. In particular, antigen-binding fragments include the well-known active fragments F(ab′)₂, Fab, Fd and Fv of an antibody. These terms are used consistently with their standard immunological meanings [Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)]. These fragments which lack the Fc fragment of intact antibody, clear more rapidly from the circulation, and may have less non-specific tissue binding than an intact antibody (Wahl et al., J. Nucl. Med. 24:316-325 (1983)). Methods for obtaining a single domain antibody (“Fd”) which comprises an isolated variable heavy chain single domain, have been reported (see, for example, Ward et al., Nature 341:644-646 (1989), disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, e.g., Moore et al., U.S. Pat. No. 4,462,334. Other references describing the use and generation of antibody fragments include e.g., Fab fragments (Tijssen, Practice and Theory of Enzyme Immunoassays (Elsevier, Amsterdam, 1985)), Fv fragments (Hochman et al., Biochemistry 12: 1130 (1973); Sharon et al., Biochemistry 15: 1591 (1976); Ehrilch et al., U.S. Pat. No. 4,355,023) and portions of antibody molecules (Audilore-Hargreaves, U.S. Pat. No. 4,470,925). Those skilled in the art may construct antibody fragments from various portions of intact antibodies without destroying the specificity of the antibodies for the epitope.

Antigen-binding fragments can also be or include one or more of the complementarity determining regions (CDRs) of an antibody. The CDRs directly interact with the epitope of the antigen (see, in general, Clark, 1986; Roitt, 1991). In both the heavy chain and the light chain variable regions of IgG immunoglobulins, there are four framework regions (FR1 through FR4) separated, respectively, by three complementarity determining regions (CDR1 through CDR3). The framework regions (FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions can be incorporated into other antibodies or peptides to confer the identical antigen specificity onto that antibody or peptide.

According to one embodiment, the peptide is an intact soluble monoclonal antibody in an isolated form or in a pharmaceutical preparation. An intact soluble monoclonal antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. As used herein the term “monoclonal antibody” refers to a homogenous population of immunoglobulins which specifically bind to an epitope (i.e., antigenic determinant). One of ordinary skill in the art can easily identify antibodies having the binding characteristics of the monoclonal antibodies described herein using screening and binding assays. One of ordinary skill in the art can also easily produce other antibodies that are MBL inhibitors with only routine experimentation known to those of ordinary skill in the art.

Murine monoclonal antibodies may be made by any of the methods known in the art utilizing an immunogen. An example of a method for producing murine monoclonals to MBL is the following: female Balb/C mice are initially inoculated (i.p.) with 250 ul of the following mixture: 250 μl Titermax mixed with 100 μg human MBL in 250 μl PBS. The following week and for three consecutive weeks the mice are injected with 50 μg hMBL in 250 μl PBS. On the 4th week the mice are injected with 25 μg MBL in 250 μl PBS and the mice are fused 4 days later. The fusion protocol is adapted from Current Protocols in Immunology. The splenocytes are fused 1:1 with myeloma fusion partner P301 from ATCC using PEG 150 at 50% w/v. The fused cells are plated at a density of 1.25×10⁶/m. with 100 μl/well of a 96 well microtiter plate. The fusion media can consist of Deficient DME high glucose, Pen/Strep (50,000 U pen, 50,000 μg strep per liter), 4 mM L-glutamine, 20% fetal bovine serum, 10% thyroid enriched media, 1% OPI, 1% NEAA, 1% HAT, and 50 μM 2-mercaptoethanol. The cells are fed 100 μl/well on day one and 100/well media are exchanged on days 2, 3, 4, 7, 9, 11, and 13. The last media change before primary screening can consist of HAT substituted for the 1% HT. All subsequent feedings are done with fusion media minus the minus HT or HAT. Screening is done with human MBL plated to plastic ELISA plates (96 well plates). Purified hMBL is plated in each well at 50 μl volume containing 2 μg/ml MBL in 2% sodium carbonate buffer. The plates are then blocked with 3% BSA in PBS. Tissue culture media (50 μl) is placed in the wells and incubated for 1 hour at room temperature. The plates are washed and a secondary HRP labeled goat anti-mouse IgG antibody is used for detection. Colorimetric analysis is done with ABTS and read at 405 nm. Positive controls can consist of a polyclonal antibody to human MBL. Cells are then grown in media consisting of the following: DMEM high glucose no-I-glut, sod, pyruvate 500 ml (Irvine Scientific #9024), heat inactivated Hyclone 10%, 1% Non-essential amino acids, 4 mM L-glutamine, 100 U/ml penicillin and 100 μg/ml streptomycin. All positive wells are then screened for function in a secondary screen.

Human monoclonal antibodies may be made by any of the methods known in the art, such as those disclosed in U.S. Pat. No. 5,567,610, issued to Borrebaeck et al., U.S. Pat. No. 5,65,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, New York, 1987), and Boerner et al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

In another embodiment, the peptide is an intact humanized monoclonal antibody in an isolated form or in a pharmaceutical preparation. A “humanized monoclonal antibody” as used herein is a human monoclonal antibody or functionally active fragment thereof having human constant regions and a CDR3 region from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.). European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody.

The antibody or antigen-binding fragment thereof can be a single-chain antibody or a diabody. Although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Diabodies are bivalent, bispecific antibodies in which the VH and VL domains are expressed on a single polypeptide chain, but using a linker that is too short to allow for pairing between the two domains on the same chain, thereby forcing the domains to pair with complementary domains of another chain and creating two antigen-binding sites (see e.g., Holliger, P., et al. (1993) Proc. Natl. Acad. Sci. USA 90:6444-6448; Poijak, R. J., et al. (1994) Structure 2:1121-1123).

MBL inhibitors can be synthesized or produced by recombinant means by those of ordinary skill in the art. Such methods are well known to those of ordinary skill in the art. Peptides can be synthesized for example, using automated peptide synthesizers which are commercially available. The peptides can be produced by recombinant techniques by incorporating the DNA expressing the peptide into an expression vector and transforming cells with the expression vector to produce the peptide.

The MBL inhibitors can be used alone as a primary therapy or in combination with other therapeutics as an adjuvant therapy to enhance the therapeutic benefits of other medical treatments. Therefore, in some embodiments, the methods provided include the administration of an additional therapeutic agent. The additional therapeutic agent can be administered concomitantly with, subsequently to or prior to the administration of a MBL inhibitor as provided herein. The administration can be in vivo or in vitro.

In one embodiment, the additional therapeutic agent is an agent for treating Type I diabetes. An “agent for treating Type I diabetes” includes insulin. In another embodiment, the additional therapeutic agent is an agent for treating Type II diabetes. “An agent for treating Type II diabetes” includes insulin, sulfonylureas, meglitinides, biguanides, thiazolidinediones and alpha-glucosidase inhibitors. In a further embodiment, the additional therapeutic agent is one for treating hypertrophy, such as cardiac hypertrophy. In another embodiment, the additional therapeutic agent is one for treating nonischemic hypertrophy, such as nonischemic cardiac hypertrophy. In still a further embodiment, the additional therapeutic agent is one for treating cardiomyopathy, such as nonischemic cardiomyopathy.

According to the methods of the invention, the compositions may be administered in a pharmaceutically acceptable composition. In general, pharmaceutically-acceptable carriers are well-known to those of ordinary skill in the art. As used herein, a pharmaceutically-acceptable carrier means a non-toxic material that does not interfere with the effectiveness of the biological activity of the active ingredients, i.e., the ability of the MBL inhibitor to inhibit LCP-associated complement activation and/or to effect the desired therapeutic endpoint. Pharmaceutically acceptable carriers include diluents, fillers, salts, buffers, stabilizers, solubilizers and other materials which are well-known in the art. Exemplary pharmaceutically acceptable carriers for peptides in particular are described in U.S. Pat. No. 5,211,657. The inhibitors of the invention may be formulated into preparations in solid, semi-solid, liquid or gaseous forms such as tablets, capsules, powders, granules, ointments, solutions, depositories, inhalants (e.g., aerosols) and injections, and usual ways for oral, parenteral or surgical administration. The invention also embraces locally administering the compositions of the invention, including as implants.

According to the methods of the invention the compositions can be administered by injection by gradual infusion over time or by any other medically acceptable mode. The administration may, for example, be intravenous, intraperitoneal, intramuscular, intracavity, subcutaneous or transdermal. Preparations for parenteral administration include sterile aqueous or nonaqueous solutions, suspensions and emulsions. Examples of nonaqueous solvents are propylene glycol, polyethylene glycol, vegetable oil such as olive oil, and injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation. When the compositions of the invention are administered for the treatment of pulmonary disorders the compositions may be delivered for example by aerosol.

The compositions of the invention are administered in therapeutically effective amounts. As used herein, an “effective amount” of the inhibitor of the invention is a dosage which is sufficient to inhibit the increase in, maintain or even reduce the amount of undesirable LCP-associated complement activation. The effective amount can also be one that is sufficient to produce the desired effect, which includes treating the disease, disorder or condition in a subject such that the progression of the disease, disorder or condition is halted or reversed and/or the symptoms associated with the disease, disorder or condition are ameliorated or decreased. The amount effective for the treatment of a disease, disorder or condition, in some embodiments, includes amounts that are effective in preventing the occurrence of the disease, disorder or condition in a subject. As used herein, “treat” or “treating” generally is intended to include prophylactic measures. In some embodiments, however, the treatment is not prophylactic, and such treatment is distinguished with language that specifically excludes prevention.

Generally, a therapeutically effective amount may vary with the subject's age, condition, and sex, as well as the extent of the disease in the subject and can be determined by one of skill in the art. The dosage may be adjusted by the individual physician or veterinarian in the event of any complication. A therapeutically effective amount typically will vary from about 0.01 mg/kg to about 500 mg/kg, were typically from about 0.1 mg/kg to about 200 mg/kg, and often from about 0.2 mg/kg to about 20 mg/kg, in one or more dose administrations daily, for one or several days (depending of course of the mode of administration and the factors discussed above). A concentration of the inhibitor, in one embodiment, is a concentration which is equimolar to the concentration of MBL in the plasma of a subject. The normal plasma concentration of MBL can be assessed clinically. A normal range of MBL is approximately 1-2 μg MBL/ml plasma.

One of skill in the art can determine what an effective amount of an inhibitor is by screening the ability of the inhibitor to inhibit the LCP-associated complement activation in an in vitro assay. The activity of the inhibitor can be defined in terms of the ability of the inhibitor to inhibit LCP-associated complement activation and/or its ability to effect the desired therapeutic endpoint. An assay for measuring the ability of a putative inhibitor to, for example, inhibit LCP-associated complement activation are provided herein or otherwise known to those of ordinary skill in the art. The exemplary assay provided is predictive of the ability of an inhibitor to inhibit LCP-associated complement activation in vivo and, hence, can be used to select inhibitors for therapeutic applications.

The MBL inhibitors may be administered in a physiologically acceptable carrier. The term “physiologically-acceptable” refers to a non-toxic material that is compatible with the biological systems such of a tissue or organism. The physiologically acceptable carrier must be sterile for in vivo administration. The characteristics of the carrier will depend on the route of administration. The characteristics of the carrier will depend on the route of administration.

The invention further provides detectably labeled, immobilized and toxin conjugated forms of the MBL inhibitors. For example, the inhibitors may be labeled using radiolabels, fluorescent labels, enzyme labels, free radical labels, avidin-biotin labels, or bacteriophage labels, using techniques known to the art (Chard, Laboratory Techniques in Biology “An Introduction to Radioimmunoassay and Related Techniques,” North Holland Publishing Company (1978).

Typical fluorescent labels include fluorescein isothiocyanate, rhodamine, phycoerythrin, phycocyanin, allophycocyanin and fluorescamine.

Typical chemiluminescent compounds include luminol, isoluminol, aromatic acridinium esters, imidazoles and the oxalate esters.

Typical bioluminescent compounds include luciferin and luciferase. Typical enzymes include alkaline phosphatase, β-galactosidase, glucose-6-phosphate dehydrogenase, maleate dehydrogenase, glucose oxidase and peroxidase.

In another aspect, a method is also provided for evaluating a MBL inhibitor. The method, in one embodiment, can include the steps of selecting a candidate MBL inhibitor and assessing its ability to treat hypertrophy or cardiomyopathy in a subject. The step of assessing can, in another embodiment, include administering the candidate MBL inhibitor to the subject. The subject can be any of the subjects as provided herein. In one embodiment, the subject is MBL sufficient. In another embodiment, the method includes a step of determining whether or not the subject is MBL sufficient. In one embodiment the subject is a non-human animal. The non-human animal can be a primate, dog, cat, horse, sheep, goat, cow, rabbit, pig or rodent. The step of assessing, in a further embodiment, can also include inducing hypertrophy and/or cardiac myopathy in the subject prior to administering the candidate MBL inhibitor. Cardiac hypertrophy can be induced, for example, by aortic banding, injection of microbeads into the coronary vasculature, cardiac ischemia followed by reperfusion for many weeks, isoproternol injections, etc. Other methods for inducing hypertrophy and/or cardiomyopathy are known to those of ordinary skill in the art. The methods also include those as described elsewhere herein, such as in the Examples.

The step of assessing can, in another embodiment, further include comparing the result of the administration with the outcome in a control subject. “Outcome in a control subject”, as used herein, refers to the effect of administering the candidate inhibitor to a control subject or any effect appropriate for evaluating the candidate inhibitor by comparison with the control. In some embodiments, the hypertrophy and/or cardiac myopathy is induced in more than one subject. In other embodiments, there is more than to one control subject. Results obtained can then, for example, be evaluated by those of ordinary skill in the art with routine methods or those described below in the Examples. Similar methods can also be performed for evaluating a MBL inhibitor and its effects on a subject with diabetes. Diabetes can be induced, for example, by Streptozotocin (STZ), alloxan, db/db or ob/ob genotype mice, feeding animals a Western style diet with low dose STZ (type II diabetes), etc. Other methods for inducing diabetes in a subject include those as otherwise described elsewhere herein. Still other methods are known to those of ordinary skill in the art.

In another aspect, a method of assessing the ability of a candidate MBL inhibitor to inhibit hyperglycemic myocardial damage and/or to inhibit the loss of cardiac progenitor cells is also provided. The method, in one embodiment, can include the steps of selecting a candidate MBL inhibitor, contacting the candidate MBL inhibitor with cardiac cells, and assessing its ability to inhibit hyperglycemic myocardial damage and/or to inhibit the loss of cardiac progenitor cells. The contacting, in one embodiment, is carried out by administering the candidate MBL inhibitor to a subject. In another embodiment, the subject is MBL sufficient. In yet another embodiment, the method includes a step of determining whether or not the subject is MBL sufficient. The contacting, in a further embodiment, is carried out in vitro. The step of assessing can, in one embodiment, also include inducing hyperglycemia in the subject or subjecting cardiac cells to hyperglycemic conditions prior to administering the candidate MBL inhibitor. The step of assessing, in another embodiment, can include inducing cardiac progenitor cell loss or subjecting cardiac cells to conditions that result in cardiac progenitor cell loss in the absence of the candidate MBL inhibitor prior to administering the candidate MBL inhibitor. The step of assessing, in a further embodiment, can further include comparing the result of the administration with the outcome in the control cell(s) or a control subject or subjects. Methods of producing hyperglycemic conditions and/or inducing cardiac progenitor cell loss are described herein and are otherwise known to those of ordinary skill in the art.

Still another method for evaluating a candidate MBL inhibitor, in one aspect, involves the step of contacting a mammalian cell (e.g., cardiac cell) from any of the subjects as provided herein with a MBL inhibitor and detecting the level of LCP-associated complement activation. The result can, in one embodiment, be compared with the result from the same method but using a control (e.g., a molecule that is known to not be a MBL inhibitor). A significant reduction in the level of LCP-associated complement activation with the candidate MBL inhibitor signifies that the candidate is useful in the methods provided herein.

Also included, in another aspect, is a method for screening any of the subjects as provided herein for susceptibility to treatment with a MBL inhibitor. Such a method can be accomplished, in one embodiment, by isolating a mammalian cell from the subject and detecting the presence of a MBL or a MBL ligand on the surface of the mammalian cell. The presence of the MBL indicates that the cell is susceptible to LCP-associated complement activation, and that the subject is susceptible to treatment with a MBL inhibitor. The mammalian cell can be isolated by any method known in the art, for instance, by a biopsy. Another method for accomplishing the screening assay, in one embodiment, involves the steps of contacting a mammalian cell from the subject with a labeled isolated MBL inhibitor and detecting the presence of a MBL on the surface of the mammalian cell.

These assays can be performed in vitro, ex vivo, or in vivo. Many labels which can be used to observe the MBL inhibitor interacting with the mammalian cell are known in the art under each of these conditions. For instance, radioactive compounds can be used in vitro, and other biocompatible labels can be used ex vivo or in vivo. Once the subjects are identified which are susceptible to treatment with a MBL inhibitor, the subjects can then be treated according to the methods of the invention.

A “subject”, as used herein, includes humans, non-human primates, dogs, cats, horses, sheep, goats, cows, rabbits, pigs and rodents.

The following examples are provided to illustrate specific instances of the practice of the present invention and are not to be construed as limiting the present invention to these examples. As will be apparent to one of ordinary skill in the art, the present invention will find application in a variety of compositions and methods.

### EXAMPLES

### Methods

All animals used in MI/R experiments were male mice aged 8-12 wk old. C57BL/6 [wildtype (WT)] mice were obtained from Charles River Laboratories (Wilmington, Mass.). MBL null animals 21, back-crossed 8-10 generations onto the C57BL/6 background, were used as previously described^{2,22}. All procedures were reviewed and conducted in accordance with the Institute's Animal Care and Use Committee (IACUC). Experiments were performed according to the standards and principles set forth in the National Institutes of Health (Guide for the Care and Use of Laboratory Animals-DHHS publication no. (NIH) 85-23, revised 1985). Mice were made diabetic by a single injection of freshly prepared streptozotocin (STZ) solution (200 mg/kg body weight in citrate saline, pH 4.2, i.p., ALEXIS, Lausen, Switzerland)²³. The mice were housed in cages of four mice each and had unlimited access to water and standard mouse chow. Three days after STZ injection and immediately before MI/R studies, the urine glucose was assessed with Glucostix (Diastix, Bayer, Elkhart, Ind.). Mice with urine glucose>500 mg/dl in both tests were presumed to be diabetic. A group of STZ-injected mice was intensively treated with Lantus insulin (Sanofi-Aventis, Bridgewater, N.J.) to rule out any potentially cardiotoxic effects of STZ. Dependent on the individual daily urine glucose, 2-6 IU of insulin was injected (i.p.) daily. The insulin treatment was started on the third day post STZ injection and lasted for 2 weeks.

Experimental MI/R was performed after 14 days of a diabetic state. Diabetic and nondiabetic mice were anesthetized initially with sodium pentobarbital (60 mg/kg, i.p.), intubated, then ventilated with positive pressure on a small animal ventilator (Model 683, Harvard Apparatus, Holliston, Mass.), and anesthesia was maintained with isoflurane (1-3 MAC). After creating a longitudinal incision through the skin of the lateral left chest, the overlying chest muscles were retracted using 5-0 black-braided silk suture (Ethicon, Somerville, N.J.). The chest was opened within the third intercostal space, and the chest wall retracted using 5-0 blackbraided silk suture. An 8-0 black-braided silk suture (U.S. Surgical, Norwalk, Conn.) was passed underneath the left anterior descending coronary artery (LAD) 2 mm from the tip of the left atrium. A 1- to 2-mm piece of 0-0 suture (Deknatel, Fall River, Mass.) was placed on the LAD, and the suture tightened to occlude the artery. After 15 or 30 min of ischemia, the ligation was loosened and the 0-0 suture removed. The chest wall was closed using 5-0 black-braided silk suture (Ethicon, Somerville, N.J.). The overlying chest muscles were allowed to retract, and the skin was sutured using 5-0 black-braided silk suture (Ethicon, Somerville, N.J.). The animal was allowed to reperfuse for 4 h. An electrocardiogram (modified lead III) was evaluated before, during and after ischemia and used to establish comparable ischemia and reperfusion in every mouse. Only mice demonstrating increased ST elevation (>4 mm) during ischemia were included in the study.

Echocardiography was performed after mice had underwent experimental MI/R as described above. It has been shown that in the mouse model of MI/R, myocardial damage via infarct analysis is directly correlated with loss of function as measured by echocardiography². Therefore, echocardiography measurements were used to assess cardiac function. Echocardiography was performed 4 h after reperfusion using a Philips Sonos 5500 (Philips Medical Systems, Bothell, Wash.) with a 712 MHz animal transducer (Agilent Technologies, Santa Clara, Calif.), as described². Ejection fraction (EF) was calculated both via left ventricular M-mode measurements and via long axis length and short axis area measurements of the left ventricle (LV)^{24,25}. For EF, only M-mode data are presented, as both methods of EF measurements produced identical results. In some animals, long axis and short axis area measurements of the LV were assessed prior to MI/R to assess myocardial dimensions under nonischemic conditions.

To collect blood and tissue following reperfusion and echocardiography, the chest cavity was opened, the inferior caval vein was cut, and blood was collected from the thoracic cavity. Hearts were excised and fixed in 10% formalin PBS at 4° C. overnight. In some animals that did not undergo MI/R, heart weight and body weight were assessed in non-diabetic and diabetic animals at 10 weeks of age. In diabetic animals, diabetes was induced earlier at 8 weeks of age. Serum troponin I concentrations were measured as an index of myocardial cell death using a commercially available ELISA kit (Life Diagnostics, West Chester, Pa.)²⁶.

To stain cardiac tissue for neutrophils, excised hearts were fixed in 10% formalin PBS at 4° C. overnight. The samples were dehydrated, embedded in paraffin and cut into 7 μm sections. To evaluate neutrophil infiltration into the heart, sections were dewaxed with EZ-DeWax Solution (BioGenex, San Ramon, Calif.) and incubated with blocking buffer containing 5% normal sheep serum for 1-2 h at room temperature. Primary antibody incubation was performed with purified rat anti-mouse Ly-6G monoclonal Ab (BD Pharmingen, Franklin Lakes, N.J.) for 1-2 h at room temperature²⁷. Following the primary antibody incubation, slides were washed four times for 15 min with TBS-Tween each and incubated with a sheep anti-rat IRDye800 Ab (Rockland Immunochemicals, Gilbertsville, Pa.) for 1-2 h at room temperature. After washing again four times for 15 min each, excess fluid was removed and slide covered with Gel Mount (Biomeda, Foster City, Calif.) cover slips (Fisher Scientific, Pittsburgh, Pa.) and sealed after 1 h with clear nail polish. Finally, sections were scanned for neutrophil infiltration with an Odyssey infrared imaging system (LI-COR, Lincoln, Nebr.) at 800 nm.

Patients (n=747) with acute MI from TIMI 14 and ENTIRE-TIMI 23 trials^{28,29} were analyzed for MBL concentrations using a novel functional MBL assay³⁰. Diabetic status was evaluated and the patients broken into diabetic and non-diabetic groups. Inclusion criteria for both trials included patients aged 18 to 75 years presenting with ST-segment elevation MI who were within 6 hours of symptom onset and eligible for fibrinolytic therapy. Exclusions relevant to analysis included presentation with cardiogenic shock and suspected cocaine or amphetamine induced MI.

To visualize cardiac progenitor cell (CPC) numbers, heart sections were prepared, dewaxed and stained for the CPC-specific marker SCF R/c-kit as previously described³¹. Following incubation with a blocking buffer containing 5% normal donkey serum for 1-2 h at room temperature, sections were incubated with purified goat anti-mouse SCF R/c-kit polyclonal Ab (R&D Systems, Minneapolis, Minn.) for 1-2 h at room temperature. Following the primary antibody incubation, slides were washed four times for 15 min with TBS-Tween each and incubated with a purified donkey anti-goat IRDye800 Ab (Rockland) for 1-2 h at room temperature. After washing and blocking four times for 15 min each, excess fluid was removed and slide covered with Gel Mount (Biomeda, Foster City, Calif.) cover slips (Fisher Scientific, Pittsburgh, Pa.) and sealed after 1 h with clear nail polish. Sections were scanned for CPC with an Odyssey infrared imaging system (LI-COR, Lincoln, Nebr.) at 800 nm.

All statistical analyses of data were performed using SigmaStat software (SPSS, Chicago, Ill.). All data were evaluated using one-way ANOVA and post hoc analysis using the Student-Newman-Keuls method. MBL concentrations for TIMI trials were evaluated with a Wilcoxon Rank Sums test. All data are expressed as the mean±SE.

### Results

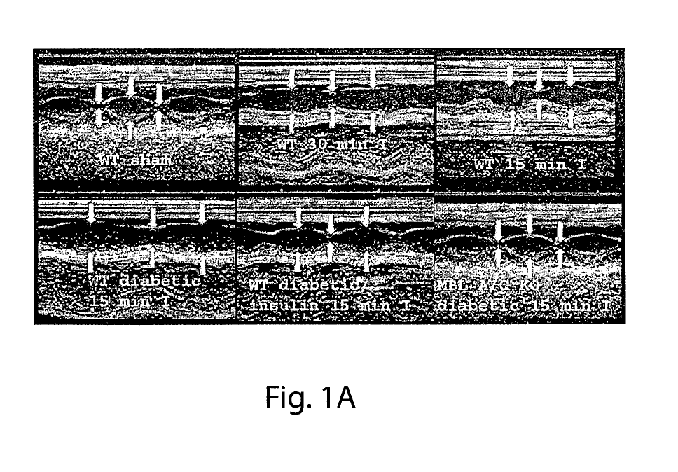

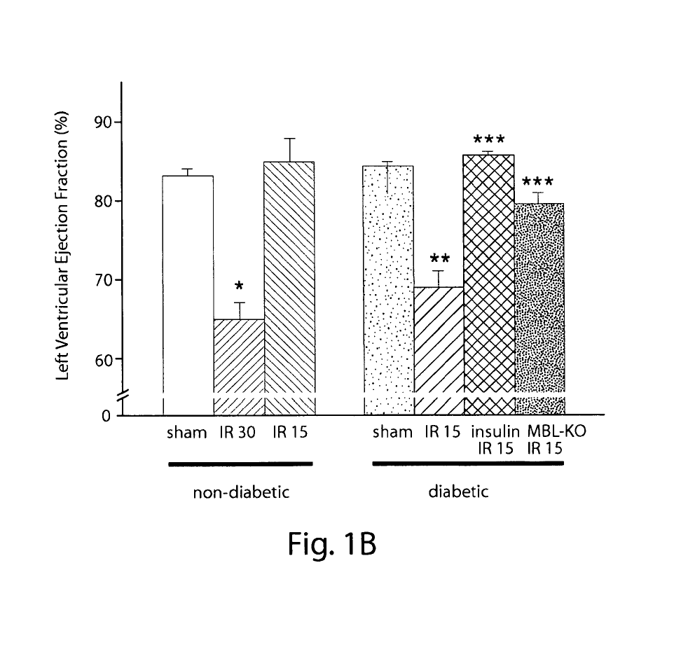

Following 30 min of ischemia and 4 h of reperfusion in non-diabetic WT mice a significant decrease in left ventricular ejection fraction (EF) compared to sham-operated animals was observed (FIG. 1). EF before MI/R in non-diabetic WT mice was comparable to sham operated animals. Increasing the ischemic time to more than 30 min (Le, up to 1 h) increased mortality, but did not increase loss of function as measured by echocardiography in surviving mice after MI/R. Thus, in this model of MI/R, 30 min of ischemia is associated with maximal loss of myocardial function and maximal myocardial injury². In non-diabetic WT mice, 15 min of ischemia and 4 h of reperfusion did not induce a significant decrease in EF compared to pre-MI/R animals and sham-operated animals (FIG. 1). Therefore, diabetic animals underwent 15 min of ischemia to investigate the effects of diabetes and hyperglycemia on MI/R related injury. The EF of sham-operated diabetic WT mice was comparable to the EF of pre MI/R, sham-operated and pre MI/R EF of nondiabetic WT mice (FIG. 1).

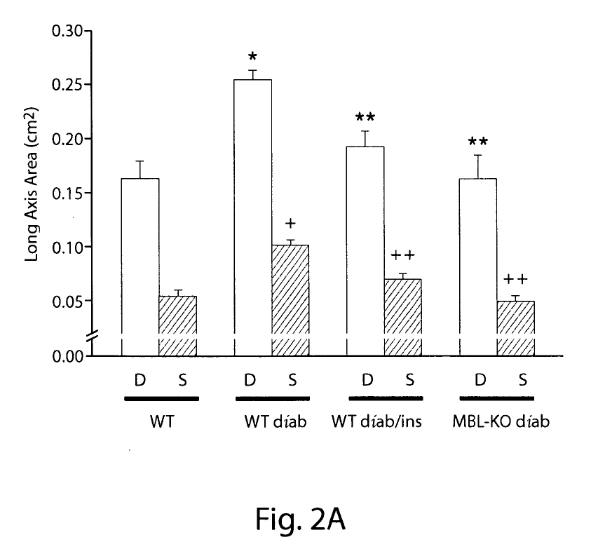

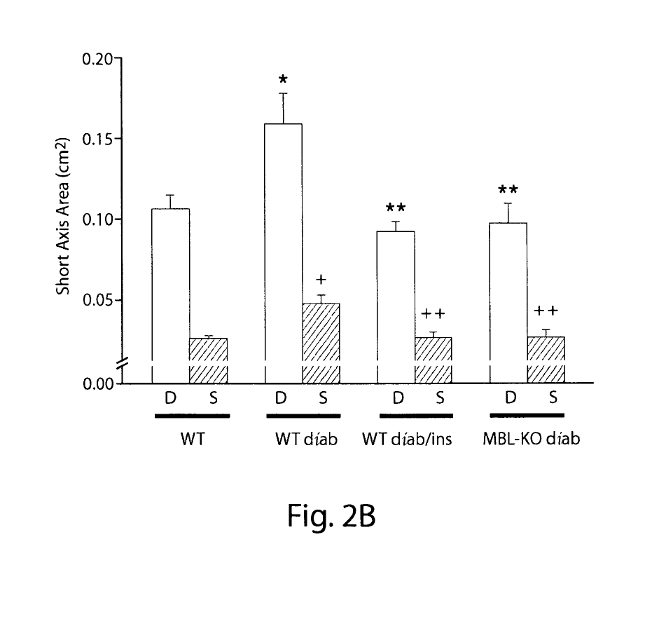

After two weeks of diabetes, mice demonstrated significantly increased long and short axis area measurements during systole and diastole by echocardiography pre-MI/R compared to non-diabetic animals (FIG. 2). However, the EF in sham-operated diabetic animals was not significantly different from non-diabetic sham-operated mice (FIG. 1B). Since EFs represent the percentage of diastolic volume that is ejected from the heart during systole, these data suggest that diastolic and systolic volumes in diabetic mice must have increased proportionally. Thus, the percentage of ejected volume in diabetic mice is comparable to non-diabetic mice, while the total ejected volume is increased. Dilated hearts with “normal” EFs suggest a state of compensated dilative diabetic cardiomyopathy.

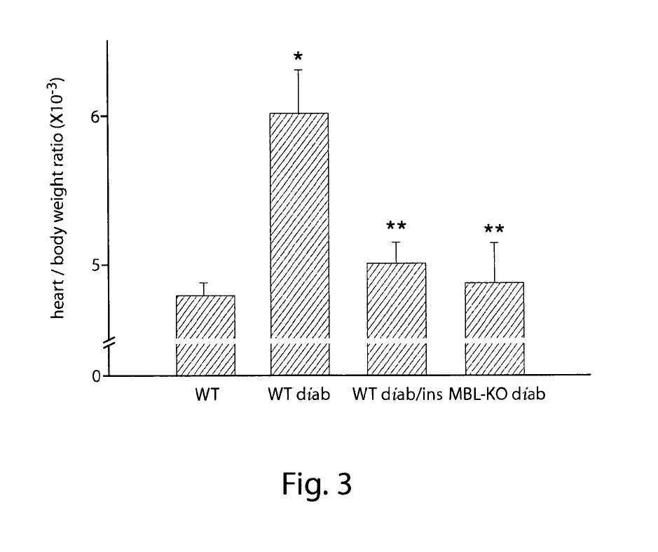

Heart/body weight ratios after 2 weeks of diabetes were significantly increased in WT mice compared to non-diabetic controls, indicating hypertrophic remodeling of the diabetic hearts (FIG. 3).

In contrast to non-diabetic mice, MI/R in diabetic animals with 15 min of ischemia induced a significant decrease in EF compared to sham-operated WT mice (FIG. 1B). These results confirm increased susceptibility to MI/R injury during diabetes and indicate MI/R induced changes from a compensated state to a decompensated state of diabetic cardiomyopathy. Insulin treatment of diabetic mice prevented loss of cardiac function following MI/R, ruling out any cardiotoxic effects of STZ (FIG. 1B). Additionally, insulin treatment protected diabetic WT mice from diabetic cardiomyopathy as measured by long and short axis measurements via echocardiography (FIG. 2), as well as heart/body weight ratios (FIG. 3). Diabetic MBL A/C KO mice were significantly protected from loss of myocardial function following MI/R compared to diabetic WT mice (FIG. 1). Diabetic MBL A/C KO mice were also protected from diabetic dilatative cardiomyopathy and hypertrophic remodeling (FIGS. 2 and 3).

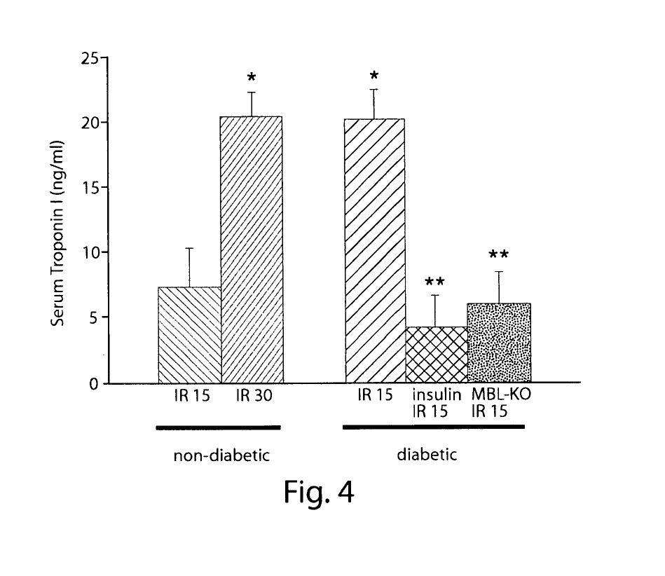

Serum troponin I concentrations provided biochemical data that confirmed and supported the echocardiographic measurements. Non-diabetic WT sham-operated animals did not present detectable concentrations of serum troponin I. Thirty min of ischemia and 4 h of reperfusion in non-diabetic WT mice significantly increased serum troponin I concentrations compared to only 15 min of ischemia followed by reperfusion (FIG. 4). In comparison to nondiabetic WT mice, diabetic WT mice had significantly increased serum troponin I concentrations following MI/R (FIG. 4). These data extend our EF observations and suggest increased susceptibility to MI/R injury in diabetic mice. Serum troponin I concentrations after MI/R were significantly reduced in insulin-treated diabetic animals compared to non-treated diabetic WT mice (FIG. 4). Confirming our echocardiographic findings, diabetic MBL A/C KO mice were significantly protected from MI/R injury following MI/R (FIG. 4).

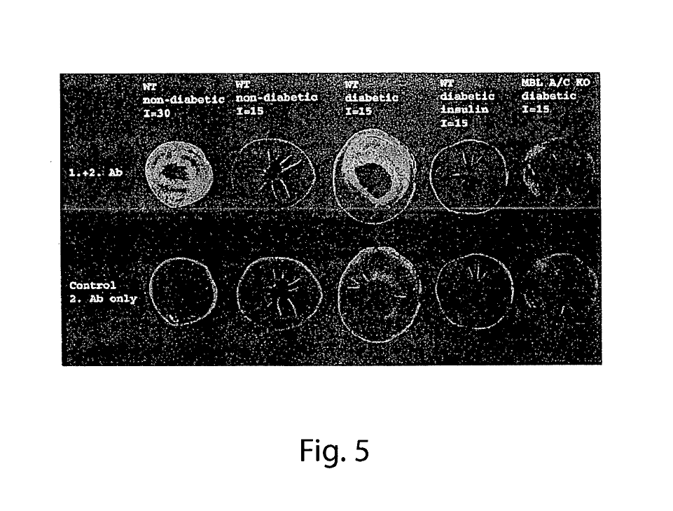

Neutrophil infiltration into the cardiac tissue as a measure of MI/R related inflammation mirrored the echocardiography and serum troponin I measurements (FIG. 5). In tissue samples from non-diabetic WT mice undergoing 15 min of ischemia and 4 h of reperfusion, no significant neutrophil infiltration was observed. In contrast, 30 min of ischemia induced a significant inflammatory response (FIG. 5). When diabetic mice were subjected to 15 min of ischemia and 4 h of reperfusion, a significant inflammatory response was observed, similar to that observed following 30 min of ischemia in non-diabetic mice. Treatment of diabetic WT mice with insulin attenuated the inflammatory response. Similar to what has been observed in non-diabetic mice², diabetic MBL A/C KO mice revealed reduced inflammatory infiltrates (FIG. 5).

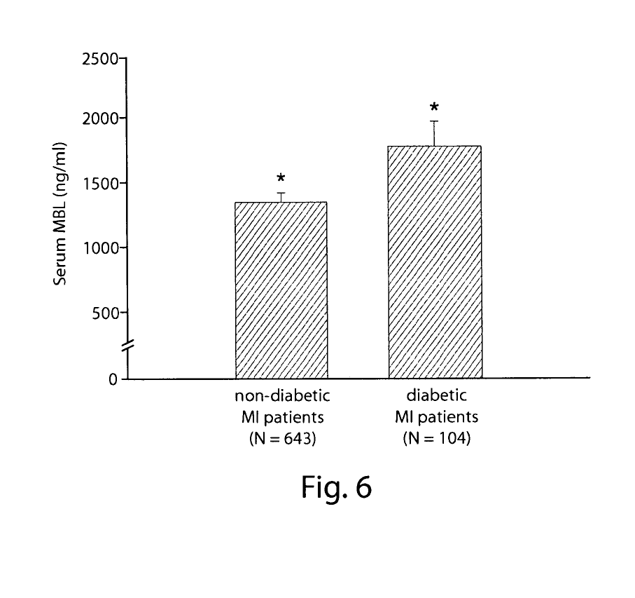

MBL concentrations in 747 patients with acute MI from TIMI 14 and ENTIRE-TIMI 23 clinical trials support the findings. Diabetics had significantly higher MBL concentrations than non-diabetic patients (FIG. 6).

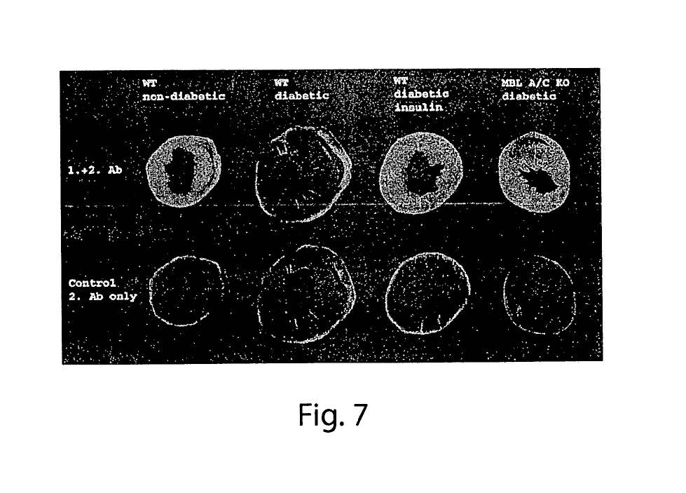

Staining for CPC revealed significantly lower numbers of CPC in diabetic WT mice compared to non-diabetic WT mice (FIG. 7). Treatment with insulin reversed this effect in the diabetic mice. Interestingly, diabetic MBL A/C KO mice were protected from loss of CPC even though insulin therapy was not given to this group (FIG. 7).

### Discussion

Using STZ-induced type I diabetic WT and MBL A/C KO mice, the contribution of MBL and the lectin complement pathway in diabetic injury, diabetic cardiomyopathy and hyperglycemic induced loss of CPC was studied.

Echocardiographic analysis of left ventricular myocardial function following MI/R demonstrated increased susceptibility to MI/R induced injury in WT mice during diabetes. Troponin I concentrations and neutrophil infiltration into the myocardium mirrored the echocardiographic results.

Diabetic MBL A/C KO mice were significantly protected from MI/R injury, while having a functional classical pathway. These findings demonstrate that MBL and lectin complement pathway activation play a role in increased susceptibility to MI/R injury during diabetes, suggesting at the same time a minor role for the classical complement pathway in diabetic MI/R injury.

Earlier studies suggested that cardiotoxic effects of STZ are due to insulin deficiency and not to a primary cardiotoxic effect of STZ³². Supporting these results, insulin treatment attenuated the myocardial injury observed in STZ induced diabetes, ruling out a significant cardiotoxic effect of STZ.

Elevated plasma glucose in MI is not only restricted to diabetic patients, but is observed as a common feature in non-diabetic MI patients³³. Recent studies suggest that admission hyperglycemia may be a more important determinant for mortality and poor prognosis after MI than a history of diabetes^{34,35}. Some studies interpret increased plasma glucose levels in MI in non-diabetic patients as relative insulin deficiency due to stress hyperglycemia³⁶, while other studies suggest that hyperglycemia at admission during an MI could represent pre-existing undiagnosed diabetes rather than stress hyperglycemia³⁷.

Since EFs were not significantly different before MI/R in diabetic groups compared to non-diabetic groups, diastolic and systolic volumes must have increased proportionally in diabetic mice. These data suggest a dilative diabetic cardiomyopathy in a compensated state. In addition, increased heart/body weight ratios in diabetic WT mice indicate hypertrophic remodeling. Thus, it is believed that hyperglycemic myocardial damage and remodeling during the two weeks of diabetes is responsible for the increased susceptibility towards enhanced MI/R injury in diabetes rather than acute hyperglycemia at the time of MI/R, causing a change from a compensated state to a decompensated state of diabetic cardiomyopathy.

Diabetes is known to cause cardiomyopathy, but the etiology is poorly understood. Enhanced oxidative stress in STZ-induced diabetes has been linked to a loss of cardiac progenitor cells (CPC) impairing regeneration of myocytes³¹. The data demonstrate and confirm that diabetic WT mice lose significant numbers of CPC following two weeks of diabetes. Since MBL A/C KO mice were protected from loss of CPC following two weeks of diabetes, the findings suggest a significant role of MBL in the hyperglycemic induced loss of CPC. Oxidative stress, which occurs in diabetes and known to play a role in CPC loss³¹, has been shown to induce lectin pathway activation³⁸. Thus, hyperglycemia in diabetes may induce MBL dependent lectin complement pathway mediated inflammation, resulting in formation of reactive oxygen species and in subsequent loss of CPC. The decreased numbers of CPC in diabetic WT hearts, resembling a decreased regenerative capability of the myocardium, may play an important role in hypertrophic remodeling, dilative cardiomyopathy and increased susceptibility to MI/R in diabetes.

The results obtained described herein using MBL null mice combined with clinical findings in patients with acute MI confirm a significant role for MBL in diabetic injury, including hyperglycemic myocardial damage, loss of CPC, remodeling and cardiomyopathy.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

### REFERENCES

The listing of the references in the following list, or as elsewhere cited herein, is not intended to be an admission that any of the references is a prior art reference.
1. Weisman H F, Bartow T, Leppo M K, Marsh H C, Jr., Carson G R, Concino M F, Boyle M P, Roux K H, Weisfeldt M L, Fearon D T. Soluble human complement receptor type 1: in vivo inhibitor of complement suppressing post-ischemic myocardial inflammation and necrosis. Science. 1990; 249:146-151.
2. Walsh M C, Bourcier T, Takahashi K, Shi L, Busche M N, Rother R P, Solomon S D, Ezekowitz R A, Stahl G L. Mannose-binding lectin is a regulator of inflammation that accompanies myocardial ischemia and reperfusion injury. J Immunol. 2005; 175:541-546.
3. Hill J H, Ward P A. The phylogistic role of C3 leukotactic fragments in myocardial infarcts of rats. J Exp Med. 1971; 133:885-900.
4. Pemberton M, Anderson G, Vetvicka V, Justus D E, Ross G D. Microvascular effects of complement blockade with soluble recombinant CR1 on ischemia/reperfusion injury of skeletal muscle. J Immunol. 1993; 150:5104-5113.
5. Wada K, Montalto M C, Stahl G L. Inhibition of Complement C5 Reduces Local and Remote Organ Injury After Intestinal Ischemia/Reperfusion in the Rat. Gastroenterology. 2001; 120:126-133.
6. Stahl G L, Xu Y, Hao L, Miller M, Buras J A, Fung M, Zhao H. Role for the Alternative Complement Pathway in Ischemia/Reperfusion Injury. Am J Pathol. 2003; 162:449-455.
7. Buerk e M, Murohara T, Lefer A M. Cardioprotective effects of a C1 esterase inhibitor in myocardial ischemia and reperfusion. Circulation. 1995; 91:393-402.
8. Horstick G, Heimann A, Gotze O, Hafner G, Berg O, Boehmer P, Becker P, Darius H, Rupprecht H J, Loos M, Bhakdi S, Meyer J, Kempski O. Intracoronary application of Clesterase inhibitor improves cardiac function and reduces myocardial necrosis in an experimental model of ischemia and reperfusion. Circulation. 1997; 95:701-708.
9. Collard C D, Vakeva A, Morrissey M A, Agah A, Rollins S A, Reenstra W R, Buras J A, Meri S, Stahl G L. Complement Activation after Oxidative Stress: Role of the Lectin Complement Pathway. Am J Pathol. 2000; 156:1549-1556.
10. Jordan J E, Montalto M C, Stahl G L. Inhibition of Mannose-Binding Lectin Reduces Postischemic Myocardial Reperfusion Injury. Circulation. 2001; 104:1413-1418.
11. McMullen M E, Hart M L, Walsh M C, Buras J, Takahashi K, Stahl G L. Mannose-binding lectin binds IgM to activate the lectin complement pathway in vitro and in vivo. Immunobiology. 2006; 211:759-766.
12. Zhang M, Takahashi K, Alicot E M, Vorup-Jensen T, Kessler B, Thiel S, Jensenius J C, Ezekowitz R A, Moore F D, Carroll M C. Activation of the lectin pathway by natural IgM in a model of ischemia/reperfusion injury. J Immunol. 2006; 177:4727-4734.
13. Greer J J M, Ware D P, Lefer D J. Myocardial infarction and heart failure in the db/db diabetic mouse. Am J Physiol Heart Circ Physiol. 2006; 290:HI46-H153.
14. Dandona P, Thusu K, Cook S, Snyder B, Makowski J, Armstrong D, Nicotera T. Oxidative damage to DNA in diabetes mellitus. Lancet. 1996; 347:444-445.
15. Pozo-Navas B, Stessel H, Wolkart G, Brunner F. Role of Myocardial Nitric Oxide in Diabetic Ischemia-Reperfusion Dysfunction: Studies in Mice with Myocyte-Specific Overexpression of Endothelial Nitric-Oxide Synthase. J Pharmacol Exp Ther. 2006; 319:729-738.
16. Haffner S M, Lehto S, Ronnemaa T, Pyorala K, Laakso M. Mortality from coronary heart disease in subjects with type 2 diabetes and in nondiabetic subjects with and without prior myocardial infarction. N Engl J. Med. 1998; 339:229-234.
17. Jacobs J, Sena M, Fox N. The cost of hospitalization for the late complications of diabetes in the United States. Diabet Med. 1991; 8 Spec No: S23-S29.
18. Malmberg K, Yusuf S, Gerstein H C, Brown J, Zhao F, Hunt D, Piegas L, Calvin J, Keltai M, Budaj A, Investigators ftOR. Impact of Diabetes on Long-Term Prognosis in Patients With Unstable Angina and Non-Q-Wave Myocardial Infarction: Results of the OASIS (Organization to Assess Strategies for Ischemic Syndromes) Registry. Circulation. 2000; 102:1014-1019.
19. Jelesoff N E, Feinglos M, Granger C B, Califf R M. Outcomes of diabetic patients following acute myocardial infarction: a review of the major thrombolytic trials. Coron Artery Dis. 1996; 7:732-743.
20. Hovind P, Hansen T K, Tarnow L, Thiel S, Steffensen R, Flyvbjerg A, Parving H H. Mannose-binding lectin as a predictor of microalbuminuria in type 1 diabetes: an inception cohort study. Diabetes. 2005; 54:1523-1527.
21. Shi L, Takahashi K, Dundee J, Shahroor-Karni S, Thiel S, Jensenius J C, Gad F, Hamblin M R, Sastry K N, Ezekowitz R A. Mannose-binding Lectin-deficient Mice Are Susceptible to Infection with Staphylococcus aureus. J Exp Med. 2004; 199:1379-1390.
22. Hart M L, Ceonzo K A, Shaffer L A, Takahashi K, Rother R P, Reenstra W R, Buras J A, Stahl G L. Gastrointestinal ischemia-reperfusion injury is lectin complement pathway dependent without involving C1q. J Immunol. 2005; 174:6373-6380.
23. Hu Y, Belke D, Suarez J, Swanson E, Clark R, Hoshijima M, Dillmann W H. Adenovirus Mediated Overexpression of O-GlcNAcase Improves Contractile Function in the Diabetic Heart. Circ Res. 2005; 96:1006-1013.
24. Sahn D J, DeMaria A, Kisslo J, Weyman A. Recommendations regarding quantitation in M mode echocardiography: results of a survey of echocardiographic measurements. Circulation. 1978; 58:1072-1083.
25. Kenchaiah S, Pfeffer M A, John Sutton M, Plappert T, Rouleau J L, Lamas G A, Sasson Z, Parker J O, Geltman E M, Solomon S D. Effect of antecedent systemic hypertension on subsequent left ventricular dilation after acute myocardial infarction (from the Survival and Ventricular Enlargement trial). The American Journal of Cardiology. 2004; 94:1-8.
26. Tao L, Liu H R, Gao F, Qu Y, Christopher T A, Lopez B L, Ma X L. Mechanical traumatic injury without circulatory shock causes cardiomyocyte apoptosis: role of reactive nitrogen and reactive oxygen species. Am J Physiol Heart Circ Physiol. 2005; 288:H2811-H2818.
27. Fleming T J, Malek T R. Multiple glycosyl phosphatidylinositol-anchored Ly-6 molecules and transmembrane Ly-6E mediate inhibition of IL-2 production. J Immunol. 1994; 153:1955-1962.
28. Antman E M, Giugliano R P, Gibson C M, McCabe C H, Coussement P, Kleiman N S, Vahanian A, Adgey A A J, Menown I, Rupprecht H J, Van der Wieken R, Ducas J, Scherer J, Anderson K, Van de Werf F, Braunwald E. Abciximab Facilitates the Rate and Extent of Thrombolysis: Results of the Thrombolysis In Myocardial Infarction (TIMI) 14 Trial. Circulation. 1999; 99:2720-2732.
29. Antman E M, Louwerenburg H W, Baars H F, Wesdorp J C L, Hamer B, Bassand J P, Bigonzi F, Pisapia G, Gibson C M, Heidbuchel H, Braunwald E, Van de Werf F, for the ENTIRE-TIMI. Enoxaparin as Adjunctive Antithrombin Therapy for ST-Elevation Myocardial Infarction: Results of the ENTIRE-Thrombolysis in Myocardial Infarction (TIMI) 23 Trial. Circulation. 2002; 105:1642-1649.
30. Walsh M C, Shaffer L A, Guikema B J et al. Fluorochrome-linked immunoassay for functional analysis of the mannose binding lectin complement pathway to the level of C3 cleavage. Journal of Immunological Methods. In press 2007.
31. Rota M, LeCapitaine N, Hosoda T, Boni A, De Angelis A, Padin-Iruegas M E, Esposito G, Vitale S, Urbanek K, Casarsa C, Giorgio M, Luscher T F, Pelicci P G, Anversa P, Leri A, Kajstura J. Diabetes Promotes Cardiac Stem Cell Aging and Heart Failure, Which Are Prevented by Deletion of the p66shc Gene. Circ Res. 2006; 99:42-52.
32. Ramanadham S, Young J, Teener T E, Jr. Prevention of streptozotocin-induced alterations in the rat heart by 3-O-methyl glucose and insulin treatments. J Cardiovasc Pharmacol. 1987; 9:291-297.
33. Sewdarsen M, Jialal I, Vythilingum S, Govender G, Rajput M C. Stress hyperglycaemia is a predictor of abnormal glucose tolerance in Indian patients with acute myocardial infarction. Diabetes Research (Edinburgh, Lothian). 1987; 6:47-49.
34. Capes S E, Hunt D, Malmber g K, Gerstein H C. Stress hyperglycaemia and increased risk of death after myocardial infarction in patients with and without diabetes: a systematic overview. The Lancet. 2000; 355:773-778.
35. Kosiborod M, Rathore S S, Inzucchi S E, Masoudi F A, Wang Y, Havranek E P, Krumholz H M. Admission Glucose and Mortality in Elderly Patients Hospitalized With Acute Myocardial Infarction: Implications for Patients With and Without Recognized Diabetes. Circulation. 2005; 111:3078-3086.
36. Oswald G A, Smith C C, Betteridge D J, Yudkin J S. Determinants and importance of stress hyperglycaemia in non-diabetic patients with myocardial infarction. Br Med J (clip Res Ed). 1986; 293:917-922.
37. Husband D J, Alberti K G, Julian D G. “Stress” hyperglycaemia during acute myocardial infarction: an indicator of pre-existing diabetes? Lancet. 1983; 2:179-181.
38. Collard C D, Lekowski R, Jordan J E, Agah A, Stahl G L. Complement activation following oxidative stress. Mol Immunol. 1999; 36:941-948.
39. Thiel S, Frederiksen P D, Jensenius J C. Clinical manifestations of mannan-binding lectin deficiency. Molecular Immunology. 2006; 43:86-96.
40. Garred P, Strom J, Quist L, Taaning E, Madsen H O. Association of mannose-binding lectin polymorphisms with sepsis and fatal outcome, in patients with systemic inflammatory response syndrome. J Infect Dis. 2003; 188:1394-1403.
41. Keller T T, van Leuven S I, Meuwese M C, Wareham N J, Luben R, Stroes E S, Hack C E, Levi M, Khaw K T, Boekholdt S M. Serum levels of mannose-binding lectin and the risk of future coronary artery disease in apparently healthy men and women. Arterioscler Thromb Vasc Biol. 2006; 26:2345-2350.
42. Ueland T, Espevik T, Kjekshus J, Gullestad L, Omland T, Squire I B, Froland S S, Mollnes T E, Dickstein K, Aukrust P. Mannose binding lectin and soluble Toll-like receptor 2 in heart failure following acute myocardial infarction. J Card Fail. 2006; 12:659-663.
43. Rugonfalvi-Kiss S, Dosa E, Madsen H O, Endresz V, Prohaszka Z, Laid J, Karadi I, Gonczol E, Selmeci L, Romics L, Fust G, Entz L, Garred P. High Rate of Early Restenosis After Carotid Eversion Endarterectomy in Homozygous Carriers of the Normal Mannose Binding Lectin Genotype. Stroke. 2005; 36:944-948.
44. Fiane A E, Ueland T, Simonsen S, Scott H, Endresen K, Gullestad L, Geiran O R, Haraldsen G, Heggelund L, Andreassen A K, Wergeland R, Froland S, Aukrust P, Mollnes T E. Low mannose-binding lectin and increased complement activation correlate to allograft vasculopathy, ischaemia, and rejection after human heart transplantation. European Heart Journal. 2005; 26:1660-1665.
45. Madsen H O, Videm V, Svejgaard A, Svennevig J L, Garred P. Association of mannose binding-lectin deficiency with severe atherosclerosis. Lancet. 1998; 352:959-960.
46. Hansen T K, Tarnow L, Thiel S, Steffensen R, Stehouwer C D, Schalkwijk C G, Parving H H, Flyvbjerg A. Association Between Mannose-Binding Lectin and Vascular Complications in Type 1 Diabetes. Diabetes. 2004; 53:1570-1576.
47. Hansen T K, Gall M A, Tarnow L, Thiel S, Stehouwer C D, Schalkwijk C G, Parving H H, Flyvbjerg A. Mannose-binding lectin and mortality in type 2 diabetes. Arch Intern Med. 2006; 166:2007-2013.

We claim:

1. A method for inhibiting hyperglycemic myocardial damage, comprising:
contacting cardiac cells with an effective amount of a mannose binding lectin (MBL) inhibitor to inhibit lectin complement pathway (LCP)-associated complement activation so as to inhibit hyperglycemic myocardial damage.

2. The method of claim 1, wherein the contacting is carried out by administering the MBL inhibitor to a subject in need thereof.

3. The method of claim 2, wherein the subject is hyperglycemic.

4. The method of claim 2, wherein the subject has diabetes.

5. The method of claim 4, wherein the subject has Type I or Type II diabetes.

6. The method of claim 1, wherein the contacting is carried out in vitro.

7. The method of claim 1, wherein the cardiac cells are cardiac fibroblasts, endothelial cells, mast cells or vascular smooth muscle cells.

8. A method for inhibiting lectin complement pathway (LCP)-associated complement activation in a subject with nonischemic cardiac hypertrophy, comprising:
administering an effective amount of a mannose binding lectin (MBL) inhibitor to inhibit LCP-associated complement activation in the subject.

9. The method of claim 8, wherein the nonischemic cardiac hypertrophy is left ventricle hypertrophy.

10. The method of claim 8, wherein the subject with nonischemic cardiac hypertrophy is hyperglycemic.

11. The method of claim 8, wherein the subject with nonischemic cardiac hypertrophy has diabetes.

12. (canceled)

13. The method of claim 8, wherein the amount to inhibit LCP-associated complement activation is effective to treat nonischemic cardiac hypertrophy.

14. A method for inhibiting lectin complement pathway (LCP)-associated complement activation in a subject with nonischemic cardiomyopathy, comprising:
administering an effective amount of a mannose binding lectin (MBL) inhibitor to inhibit LCP-associated complement activation in the subject.

15-19. (canceled)

20. A method for inhibiting loss of cardiac progenitor cells, comprising:
contacting cardiac cells with an effective amount of a mannose binding lectin (MBL) inhibitor to inhibit cardiac progenitor cell loss.

21-27. (canceled)

28. A method for inhibiting lectin complement pathway (LCP)-associated complement activation in a nonischemic diabetic subject, comprising:
administering an effective amount of a mannose binding lectin (MBL) inhibitor to inhibit LCP-associated complement activation in the subject.

29-38. (canceled)

39. A method of evaluating a candidate MBL inhibitor, comprising:
selecting a candidate MBL inhibitor, and
assessing its ability to treat cardiac hypertrophy in a subject.

40-44. (canceled)

45. A method, comprising:
selecting a candidate MBL inhibitor, and
assessing its ability to treat cardiomyopathy in a subject.

46-50. (canceled)

51. A method, comprising:
selecting a candidate MBL inhibitor,
contacting the candidate MBL inhibitor with cardiac cells, and assessing its ability to inhibit hyperglycemic myocardial damage.

52-59. (canceled)

60. A method, comprising:
selecting a candidate MBL inhibitor,
contacting the candidate MBL inhibitor with cardiac cells, and assessing its ability to inhibit cardiac progenitor cell loss.

61-69. (canceled)

70. A method, comprising:
selecting a candidate MBL inhibitor, and
assessing its ability to inhibit LCP-associated complement activation in a subject with diabetes.

71-78. (canceled)
